(19) United States
(12) Patent Application Publication
(10) Pub. No.: US 2019/0143138 A1
(43) Pub. Date: May 16, 2019

(54) SYSTEM AND METHOD TO PROMOTE HAIR GROWTH

(71) Applicant: LaserStim, Inc., Plano, TX (US)

(72) Inventors: Kim Robin Segal, Plano, TX (US); Jeff Adelglass, Plano, TX (US); Simon Chen, Plano, CA (US)

(73) Assignee: LaserStim, Inc., Plano, TX (US)

(21) Appl. No.: 16/248,510

(22) Filed: Jan. 15, 2019

Related U.S. Application Data
(63) Continuation-in-part of application No. 16/169,301, filed on Oct. 24, 2018.
Application No. 16/169,301 is a continuation-in-part of application No. 16/123,085, filed on Sep. 6, 2018.
Application No. 16/123,085 is a continuation-in-part of application No. 16/108,424, filed on Aug. 22, 2018.
(60) Provisional application No. 62/617,416, filed on Jan. 15, 2018.
(60) Provisional application No. 62/576,441, filed on Oct. 24, 2017.
(60) Provisional application No. 62/555,040, filed on Sep. 6, 2017.
(60) Provisional application No. 62/548,401, filed on Aug. 22, 2017.

(51) Int. Cl. A61N 5/06

(52) U.S. Cl. CPC A61N 5/0617; A61B 5/448; A61N 2005/0626

(57) ABSTRACT

A device is provided which includes one or more sensors operable to measure one or more hair growth parameters of a tissue of a subject. A plurality of light sources is operable to radiate optical energy at a predetermined wavelength to promote hair growth. A control unit is coupled with the one or more sensors and the plurality of light sources, and a memory stores instructions executable by the control unit. The instructions may include to: receive, from the one or more sensors, the one or more measured hair growth parameters; determine a region within a boundary to be treated based on the one or more measured hair growth parameters; and radiate, by the plurality of light sources, optical energy at the predetermined wavelength at the region to be treated. The optical energy may be radiated within the boundaries of the region.

### CROSS-REFERENCE TO RELATED APPLICATIONS

This application (1) is a continuation-in-part of U.S. patent application Ser. No. 16/108,424 filed on Aug. 22, 2018 which claims the benefit of U.S. patent application Ser. No. 62/548,401 titled “INTERCHANGEABLE MODULAR CAP FOR LASER LIGHT THERAPY” filed on Aug. 22, 2017; (2) is a continuation-in-part of U.S. patent application Ser. No. 16/123,085 filed on Sep. 6, 2018 which claims the benefit of U.S. patent application Ser. No. 62/555,040 titled “HELMET AND MODULAR CAP FOR LASER LIGHT HAIR GROWTH THERAPY” filed on Sep. 6, 2017; (3) is a continuation-in-part of U.S. patent application Ser. No. 16/169,301 filed on Oct. 24, 2018 which claims the benefit of U.S. patent application Ser. No. 62/576,441 titled “METHOD OF COMBINING LASER LIGHT THERAPY WITH BIOACTIVE COMPOUNDS FOR PROMOTING HAIR GROWTH” filed on Oct. 24, 2017; and (4) claims the benefit of U.S. patent application Ser. No. 62/617,416 titled “APPARATUS AND METHOD FOR PROMOTING HAIR GROWTH” filed on Jan. 15, 2018, the contents of each of the above is incorporated by reference in its entirety.

### BACKGROUND

### 1. FIELD

The present description relates to optical irradiation of tissue to promote hair growth.

### 2. RELATED ART

Alopecia (hair loss) is a major concern for the adult population. Expenditures for hair restoration products and treatments for hair loss represent a major component of the multibillion-dollar cosmetic industry in the United States. Examples of techniques for hair retention and regeneration include the use of hair weaving, the use of hairpieces, the application of hair thickening sprays and shampoos, hair transplantation, and the fashioning of coiffures which distribute hair to cover balding regions of the scalp. In addition, topical drug therapies, such as Minoxidil (Rogaine®) or oral drug therapies such as Finasteride (Propecia®), are in current use to stimulate hair growth in men suffering from male pattern baldness, i.e. baldness occurring at the crown and temples. However, this chemical cannot be used by women, can cause a negative skin reaction on the scalp, and is, therefore, not suitable for everyone, and efficacy is limited and not universal.

Diode laser systems have been developed for various medical treatments of the human body. See for example, Applicant's prior U.S. Pat. Nos. 5,755,752 and 6,033,431, which are both incorporated herein by reference in their entirety. Depending on the type of treatment desired, lasers of various wavelengths, periods of exposure and other such influencing factors have been developed.

Optical energy generated by lasers has been used for various medical and surgical purposes because laser light, as a result of its monochromatic and coherent nature, can be selectively absorbed by living tissue. The absorption of the optical energy from laser light depends upon certain characteristics of the wavelength of the light and properties of the irradiated tissue, including reflectivity, absorption coefficient, scattering coefficient, thermal conductivity, and thermal diffusion constant. The reflectivity, absorption coefficient, and scattering coefficient are dependent upon the wavelength of the optical radiation. The absorption coefficient is known to depend upon such factors as interband transition, free electron absorption, grid absorption (photon absorption), and impurity absorption, which are also dependent upon the wavelength of the optical radiation.

In living tissue, water is a predominant component and has, in the infrared portion of the electromagnetic spectrum, an absorption band determined by the vibration of water molecules. In the visible portion of the spectrum, there exists absorption due to the presence of hemoglobin. Further, the scattering coefficient in living tissue is a dominant factor.

Thus, for a given tissue type, the laser light may propagate through the tissue substantially unattenuated, or may be almost entirely absorbed. The extent to which the tissue is heated and ultimately destroyed depends on the extent to which it absorbs the optical energy. It is generally preferred that the laser light be essentially transmissive through tissues which are not to be affected, and absorbed by tissues which are to be affected. For example, when applying laser radiation to a region of tissue permeated with water or blood, it is desired that the optical energy not be absorbed by the water or blood, thereby permitting the laser energy to be directed specifically to the tissue to be treated. Another advantage of laser treatment is that the optical energy can be delivered to the treatment tissues in a precise, well-defined location such as an acupuncture point and at predetermined, limited energy levels.

Ruby and argon lasers are known to emit optical energy in the visible portion of the electromagnetic spectrum, and have been used successfully in the field of ophthalmology to reattach retinas to the underlying choroidea and to treat glaucoma by perforating anterior portions of the eye to relieve interoccular pressure. The ruby laser energy has a wavelength of 694 nanometers (nm) and is in the red portion of the visible spectrum. The argon laser emits energy at 488 nm and 515 nm and thus appears in the blue-green portion of the visible spectrum. The ruby and argon laser beams are minimally absorbed by water, but are intensely absorbed by blood chromogen hemoglobin. Thus, the ruby and argon laser energy is poorly absorbed by non-pigmented tissue such as the cornea, lens and vitreous humor of the eye, but is absorbed very well by the pigmented retina where it can then exert a thermal effect.

Another type of laser which has been adapted for surgical use is the carbon dioxide (CO2) gas laser which emits an optical beam which is absorbed very well by water. The wavelength of the CO2 laser is 10,600 nm and therefore lies in the invisible, far infrared region of the electromagnetic spectrum, and is absorbed independently of tissue color by all soft tissues having a high water content. Thus, the CO2 laser makes an excellent surgical scalpel and vaporizer. Since it is completely absorbed, its depth of penetration is shallow and can be precisely controlled with respect to the surface of the tissue being treated. The CO2 laser is thus well-suited for use in various surgical procedures in which it is necessary to vaporize or coagulate neutral tissue with minimal thermal damage to nearby tissues.

Another laser in widespread use is the neodymium doped yttrium-aluminum-garnet (Nd:YAG) laser. The Nd:YAG laser has a predominant mode of operation at a wavelength of 1064 nm in the near infrared region of the electromagnetic spectrum. The Nd:YAG optical emission is absorbed to a greater extent by blood than by water making it useful for coagulating large, bleeding vessels. The Nd:YAG laser has been transmitted through endoscopes for treatment of a variety of gastrointestinal bleeding lesions, such as esophageal varices, peptic ulcers, and arteriovenous anomalies.

The foregoing applications of laser energy are thus well suited for use as a surgical scalpel and in situations where high-energy thermal effects are desired, such as tissue vaporization, tissue cauterization, and coagulation.

Although the foregoing laser systems perform well, they commonly generate large quantities of heat and require a number of lenses and mirrors to properly direct the laser light and, accordingly, are relatively large, unwieldy, and expensive. These problems are somewhat alleviated in some systems by locating a source of laser light distal from a region of tissue to be treated and providing fiber optic cable for carrying light generated from the source to the tissue region, thereby obviating the need for a laser light source proximal to the tissue region. Such systems, however, are still relatively large and unwieldy and, furthermore, are much more expensive to manufacture than a system which does not utilize fiber optic cable. Moreover, the foregoing systems generate thermal effects, which can damage living tissue, rather than provide therapeutic treatment to the tissue.

The foregoing is intended to be illustrative and is not meant in a limiting sense. Many features of the examples may be employed with or without reference to other features of any of the examples. Additional aspects, advantages, and/or utilities of the present inventive concept will be set forth in part in the description that follows and, in part, will be apparent from the description, or may be learned by practice of the present inventive concept.

### SUMMARY

The present inventive concept provides a device that overcomes the aforementioned disadvantages of conventional light emitting therapeutic devices. The device of the present inventive concept generally includes a plurality of light sources, one or more sensors, and a control unit coupled with the sensors and light sources. The sensors measure one or more hair growth parameters such as number of hair growth follicles and/or blood flow of tissue. The control unit then determines a region to be treated based on the hair growth parameters which indicate a lower hair density. The device can then focus the light sources to radiate optical energy at a predetermined wavelength at the region to be treated, for example by moving the light sources to be positioned to radiate the optical energy at the region and/or controlling the intensity and/or power of the light sources to radiate the optical energy at the region.

The aforementioned may be achieved in an aspect of the present inventive concept by providing a device. The device may include one or more sensors operable to measure one or more hair growth parameters of a tissue of a subject. A plurality of light sources may be included and operable to radiate optical energy at a predetermined wavelength to promote hair growth. A control unit may be coupled with the one or more sensors and the plurality of light sources, and a memory may store instructions executable by the control unit. The instructions may include to receive, from the one or more sensors, the one or more measured hair growth parameters. The instructions may include to determine a region within a boundary to be treated based on the one or more measured hair growth parameters, and the instructions may include to radiate, by the plurality of light sources, optical energy at the predetermined wavelength at the region to be treated. The optical energy may be radiated within the boundaries of the region.

The device may further include a plurality of modules, where each of the modules may include at least one of the plurality of light sources, and at least one of the one or more sensors. The memory may further include instructions executable by the control unit to radiate, by the light sources disposed on at least one of the plurality of modules which is positioned above the region to be treated, the optical energy at the predetermined wavelength. The device may further include a motor coupled with each of the plurality of modules, and the memory may further include instructions executable by the control unit to move at least one of the plurality of modules to position the at least one of the plurality of modules above the region to be treated. At least one of the plurality of modules may be moved by rotation around an axis. Each of the plurality of modules may further include a control unit coupled with the at least one of the plurality of light sources and the at least one of the one or more sensors, and a memory including instructions executable by the control unit. The one or more hair growth parameters may include number of hair follicles and/or blood flow. The region to be treated may be determined by the number of hair follicles being less than a predetermined number and/or blood flow less than a predetermined amount. The predetermined wavelength may be between about 100 nm and about 10,000 nm. The predetermined wavelength may be between about 550 nm and about 4000 nm.

The aforementioned may be achieved in another aspect of the present inventive concept by providing a method. The method may include measuring, by one or more sensors, one or more hair growth parameters of a tissue of a subject. A control unit may determine a region of the tissue with a boundary to be treated based on the one or more measured hair growth parameters. A plurality of light sources disposed in a device may radiate optical energy at a predetermined wavelength at the region to be treated. The optical energy may be radiated within the boundaries of the region.

The foregoing is intended to be illustrative and is not meant in a limiting sense. Many features of the embodiments may be employed with or without reference to other features of any of the embodiments. Additional aspects, advantages, and/or utilities of the present inventive concept will be set forth in part in the description that follows and, in part, will be apparent from the description, or may be learned by practice of the present inventive concept.

### BRIEF DESCRIPTION OF THE DRAWINGS

The foregoing summary, as well as the following detailed description, will be better understood when read in conjunction with the appended drawings. For the purpose of illustration, there is shown in the drawings certain examples of the present disclosure. It should be understood, however, that the present inventive concept is not limited to the precise examples and features shown. The accompanying drawings, which are incorporated in and constitute a part of this specification, illustrate an implementation of apparatuses consistent with the present inventive concept and, together with the description, serve to explain advantages and principles consistent with the present inventive concept.

FIG. 1 shows an exemplary environment of a subject with areas with less hair growth where a system can be utilized to promote hair growth in those areas;

FIG. 2A shows a schematic diagram of a diode laser irradiation system according to the present disclosure;

FIG. 2B shows an exemplary display of the system as shown in FIG. 2A;

FIG. 2C shows a diagram of an exemplary control unit which may be employed as shown in FIG. 2A;

FIG. 3 shows an cross-sectional view of an exemplary laser radiating device used in the system as shown in FIG. 2A;

FIG. 4A shows an enlarged, elevational view of a laser resonator used in the laser radiating device as shown in FIG. 3;

FIG. 4B shows an enlarged, end view of the laser resonator used in the wand as shown in FIG. 4A;

FIG. 5A shows another exemplary laser radiating device which can be used to promote hair growth;

FIG. 5B shows another exemplary laser radiating device which can be used to promote hair growth;

FIG. 5C shows another exemplary laser radiating device which can be used to promote hair growth;

FIG. 6 shows a diagram of an exemplary system to promote hair growth in accordance with the present disclosure;

FIG. 7A shows a diagrammatic view of an exemplary cap, according to examples of the present disclosure;

FIG. 7B shows a diagrammatic view of another exemplary cap illustrating the placement of the lasers for another representative example, according to examples of the present disclosure;

FIG. 8 shows a side view of the cap as shown in FIG. 7A, according to an example of the present disclosure;

FIG. 9 shows a diagrammatic plot illustrating exemplary laser placement;

FIG. 10A shows a side view of an exemplary laser diode;

FIG. 10B shows a bottom view of the exemplary laser diode as shown in FIG. 10A;

FIG. 11 shows an exemplary cap in which each laser module includes a control unit;

FIGS. 12A-12E show an exemplary interchangeable modular laser diode cap in accordance with an example of the present disclosure;

FIGS. 13A and 13B show front and side views, respectively, of an exemplary system for treating patients for hair growth stimulation, according to an example of the present disclosure;

FIGS. 14A-14D show rear, side, front and bottom views, respectively, of an exemplary light application helmet according to one example of the present disclosure;

FIGS. 15A and 15B show a top and side view, respectively, of the light application helmet shown in FIGS. 14A-14D, as it appears with the top cover removed;

FIG. 16 shows a side view of an exemplary freestanding light application device;

FIGS. 17A and 17B show diagrams of modular laser systems;

FIGS. 18A-18C show an exemplary modular laser system, according to an example of the present disclosure;

FIGS. 19A-19C show another exemplary modular laser system; and

FIG. 20 shows a flow chart of a method to promote hair growth.

### DETAILED DESCRIPTION

It is to be understood that the present inventive concept is not limited in its application to the details of construction and to the embodiments of the components set forth in the following description or illustrated in the drawings. The figures and written description are provided to teach any person skilled in the art to make and use the inventions for which patent protection is sought. The present inventive concept is capable of other embodiments and of being practiced and carried out in various ways. Persons of skill in the art will appreciate that the development of an actual commercial embodiment incorporating aspects of the present inventive concept will require numerous implementations—specific decisions to achieve the developer's ultimate goal for the commercial embodiment. While these efforts may be complex and time-consuming, these efforts, nevertheless, would be a routine undertaking for those of skill in the art of having the benefit of this disclosure. While elements may be described with one example, the elements described herein may be utilized in any suitable example in any suitable combination.

I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims. Further, it should be understood that any one of the features of the present inventive concept may be used separately or in combination with other features. Other systems, methods, features, and advantages of the present inventive concept will be, or become, apparent to one with skill in the art upon examination of the figures and the detailed description. It is intended that all such additional systems, methods, features, and advantages be included within this description, be within the scope of the present inventive concept, and be protected by the accompanying claims.

Further, any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Lastly, the terms “or” and “and/or,” as used herein, are to be interpreted as inclusive or meaning any one or any combination. Therefore, “A, B or C” or “A, B and/or C” mean any of the following: “A,” “B,” “C”; “A and B”; “A and C”; “B and C”; “A, B and C.” An exception to this definition will occur only when a combination of elements, functions, steps or acts are in some way inherently mutually exclusive.

II. General Architecture

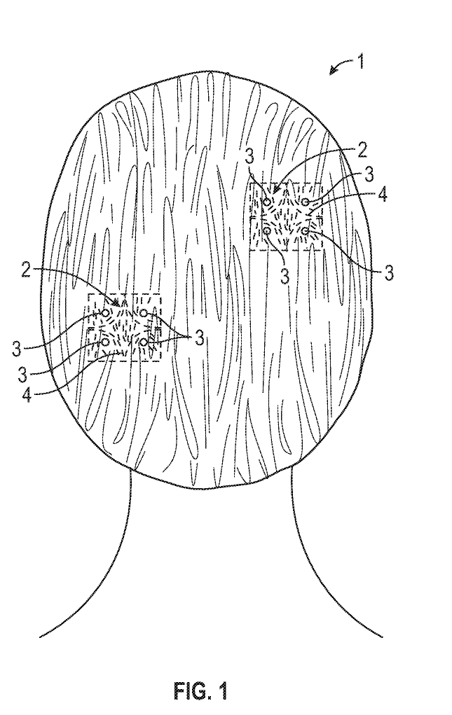

Referring to FIG. 1, a subject 1 with hair loss, for example due to alopecia or cancer treatment, is illustrated. While the rear of the head is shown, the illustration is exemplary, and the system and method discussed herein can be utilized for any area of a subject where hair growth is desired. The subject 1 has two regions 2 suffering from hair loss, or thin or lack of hair growth. The subject 1 can have one, two, three, or more regions 2 suffering from lack of hair. While the regions 2 are illustrated as rectangular, the shape of the regions 2 can be any shape and size, such as rectangular, circular, irregular, and/or triangular. The number of hair follicles 3 and/or the blood flow 4 of the subject 1 are measurements which can indicate whether the region 2 suffers from less hair density. For example, if the number of hair follicles 3 is less than other regions and/or the region 2 has less blood flow 4, then the region 2 may suffer from less hair density. Accordingly, by focusing treatment only on the regions 2 which suffer from less hair density instead of the entire head of the subject 1, the hair density can even out. During and/or after treatment, the number of hair follicles 3 and/or blood flow 4 of the regions 2 can be measured at periodic times to determine whether there is an improvement in hair growth in the regions 2.

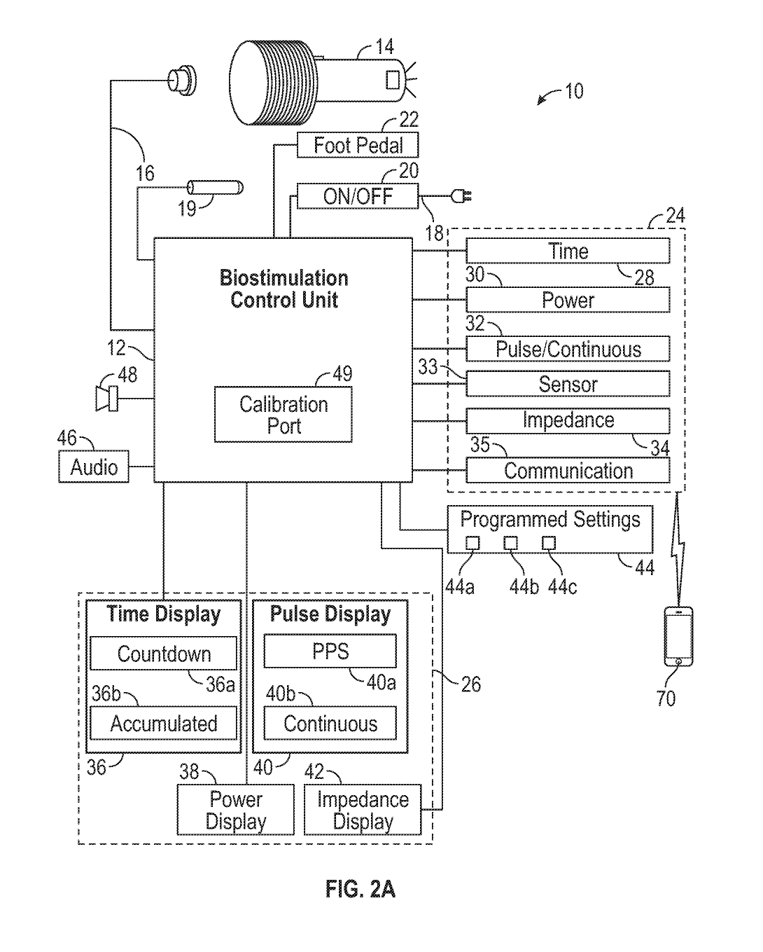

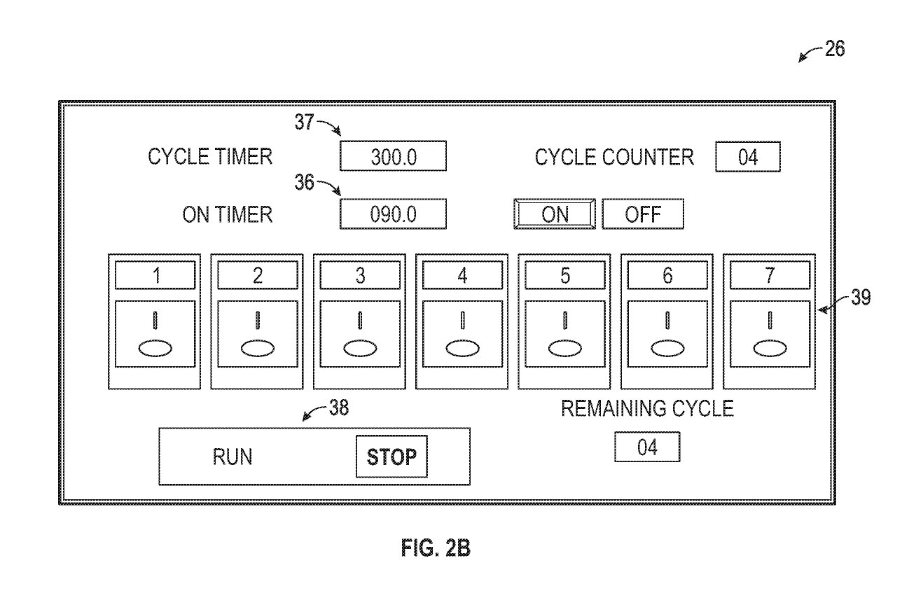

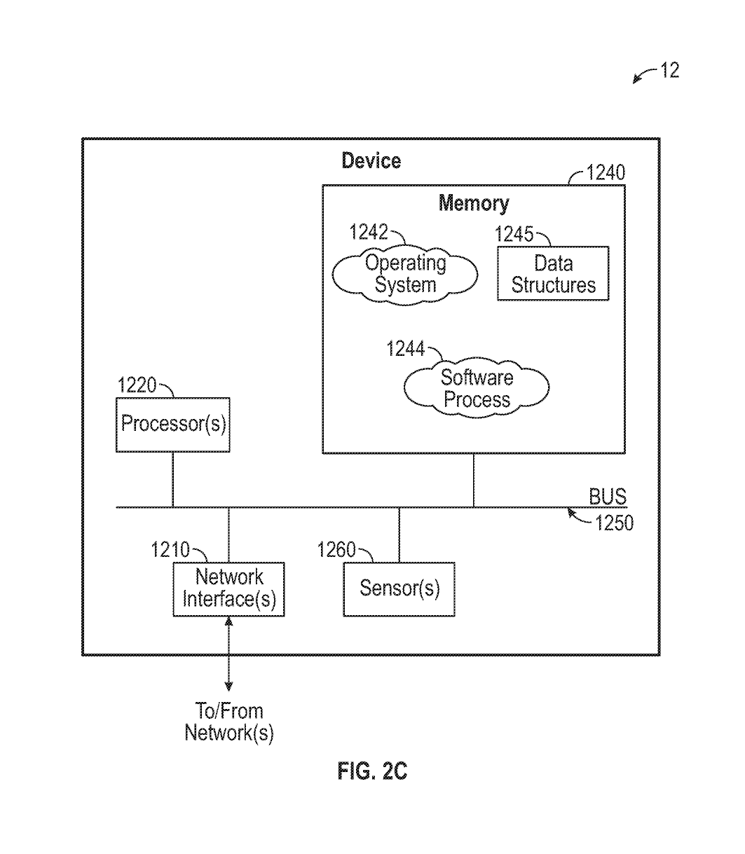

Referring to FIG. 2A, a diode laser irradiation system 10 which includes a biostimulation control unit 12 controls the operation of a laser radiating device 14, for example a hand-held laser treatment wand as illustrated in FIG. 2A. The laser radiating device 14 can be electrically connected to a control unit 12, for example via a coaxial cable 16. In other examples, the control unit 12 may be disposed within the laser radiating device 14. In yet other examples, the control unit 12 within the laser radiating device 14 may be communicatively coupled with an external device 70, for example a portable device or a computer. In at least one examples, the laser radiating device 14 may be wirelessly coupled with the external device 70, for example by wifi or Bluetooth. In some examples, the laser radiating device 14 may be coupled with the external device 70 by wire. The external device 70 may provide instructions and/or store data from the laser radiating device 14. In some examples, the external device 70 may provide data such as sensor data or patient data to the laser radiating device 14. As will be described in detail below, the laser radiating device 14 can house at least one diode laser capable of emitting optical energy, for example low level reactive laser light, for use in tissue irradiation therapy.

The control unit 12, as will also be discussed further below in FIG. 2C, can receive power through a power supply 18, for example through a power supply line adapted for connection to a conventional 120-volt power outlet. As illustrated in FIG. 2A, a ground piece 19 can be connected to the control unit 12 and/or may be held by a patient receiving the tissue irradiation therapy to provide an electrical ground for safety purposes. In other examples, a ground piece 19 is not included. An on/off switch 20 can be connected in series with the power supply 18 for controlling the flow of power to the laser radiating device 14. In at least one example, the on/off switch 20 can be on the laser radiating device 14. In some examples, the on/off switch 20 can be a separate switch external to the laser radiating device 14. In at least one example, a foot pedal 22 can be connected to the control unit 12 and is depressible to activate and/or adjust the generation and emission of laser light from the laser radiating device 14. Activation and/or adjustment may alternatively, or additionally, be provided using a switch on the laser radiating device 14.

The control unit 12 includes laser setting controls 24 and/or corresponding setting displays 26. The setting controls 24 can be utilized to select operational parameters of the control unit 12 to effect the rate of absorption and conversion to heat of tissue irradiated by the laser radiating device 14, according to desired treatment protocols. Generally, the treatment protocols provide for a rate of absorption and conversion to heat in the irradiated tissue in a range between a minimum rate sufficient to elevate the average temperature of the irradiated tissue to a level above the basal body temperature of the subject and a maximum rate which is less than the rate at which the irradiated tissue is converted to a collagenous substance. The treatment protocols vary time, power, and pulse/continuous mode parameters in order to achieve the desired therapeutic effects.

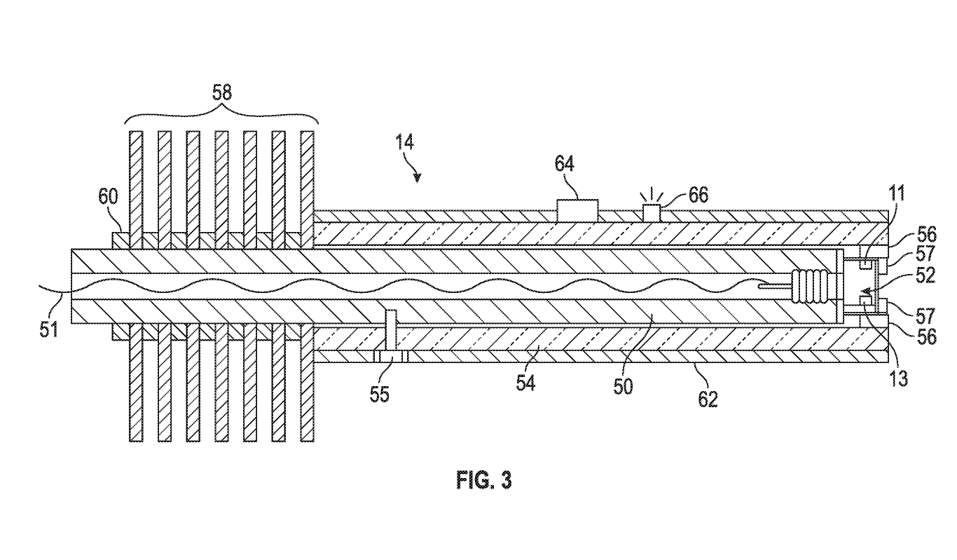

The setting controls 24 can include a treatment time control 28, a power control 30, a pulse/continuous mode control 32, sensor control 33, and/or communication control 35. Adjustments in treatment time, power and pulse/continuous mode operation of the laser radiating device 14 utilizing the controls 28-35 make possible improved therapeutic effects based upon the aforementioned treatment protocols involving one or more of these parameters. Additionally or alternately, sensor control 33 can be used to control sensors (for example hair follicle sensor 11 and/or blood flow sensor 13 as shown in FIG. 3). Also, an impedance control 34 can be provided to adjust an impedance measurement of the tissue to a baseline value, according to skin resistance, as discussed further below, whereby improvements in tissue condition may be monitored. Communication control 35 can be provided to control communication with one or more external devices 70, as discussed above. For example, communication with the one or more external devices 70 may be wireless or wired communication. It is understood that, according to the specific example of the control unit 12, the setting controls 24 may include any combination of one or more of the controls 28-35.

The setting displays 26, for example as illustrated in FIG. 2B, can include a time display 36, a power display 38, a pulse display 40, and/or an impedance display 42. In at least one example, each of the displays 26 are light emitting diode (LED) displays such that the corresponding setting controls 24 can be operated to increment or decrement the settings, which are then indicated on the displays. In some examples, the displays 26 may be combined into one display, for example with a menu option. In some examples, the displays 26 may be touch screen operated. A programmed settings control 44 can be included to save setting selections and then automatically recall them for convenience, using one or more buttons 44a-44c, for example.

The time control 28 can adjust the time that optical energy is emitted from the laser radiating device 14, as indicated on the time display 36. In at least one example, the time control 28 can control the time that the laser radiating device 14 is treating one region of the subject as well as the entire treatment. The time display 36 can include a countdown display 36a and an accumulated display 36b. Once the time control 28 is set, the countdown display 36a can indicate the setting so that as the laser radiating device 14 is operated the time is decremented to zero. The accumulated time display 36b increments from zero (or any other reset value) as the laser radiating device 14 is operated so that the total treatment time is displayed. The time display 36 takes into account the pulsed or continuous mode operation of the system 10.

The power control 30 can adjust the power dissipation level of the optical energy radiated from the laser radiating device 14. For example, when power is provided to the laser radiating device 14, the one or more diodes may radiate optical energy at a predetermined wavelength to promote hair growth. In at least one example, the predetermined wavelength can be between about 100 nm and about 10,000 nm. In some examples, the predetermined wavelength can be between about 550 and about 10,000 nm. In some examples, the predetermined wavelength can be between about 1000 and about 10,000 nm. In some examples, the predetermined wavelength can be between about 1300 and about 4000 nm. In some examples, the predetermined wavelength can be between about 1550 nm and about 3150 nm. In some examples, the predetermined wavelength can be between about 550 nm, about 635 nm, and/or about 735 nm. It is understood that other suitable wavelengths or combinations of ranges as disclosed above can be used depending on the treatment and the subject without deviating from the scope of the inventive concept. Additionally or alternately, the power control 30 can provide power to one or more other components of the laser radiating device 14.

In at least one example, the power control 30 may provide a desired amount of power to the laser radiating device 14. For example, the range of power can be from 100 to about 1500 milliwatts (mW), in some examples with operation ranging about 500 mW. The pulse/continuous mode control 32 can set the system 10 to generate laser light energy from the laser radiating device 14 either continuously or as a series of pulses. The control 32 may include, for example, a pulse duration rheostat (not shown) for adjusting the pulse-on or pulse-off time of the laser radiating device 14. In at least one example, the pulses-per-second (PPS) can be set in a range from zero to 9995, adjustable in 5 step increments. The PPS setting is displayed on a PPS display 40a. The pulse duration may alternatively, or additionally, be displayed indicating the duty cycle of pulses ranging from 5 to 99 (e.g., 5 meaning that the laser is “on” 5% of the time). A continuous mode display 40b is activated when the system 10 is being operated in the continuous wattage (CW) mode of operation.

An audio volume control 46 can be provided for generating an audible warning tone from a speaker 48 when laser light is being generated. Thus, for example, the tone may be pulsed when the system is operating in the pulse mode of operation. In at least one example, vibration may be used to alert the user of actions.

The impedance control 34 can be a sensitivity setting that is calibrated and set, according to the tissue skin resistance, to a baseline value which is then indicated on the impedance display 42. As therapy progresses the impedance readout on the display 42 changes (i.e., it decreases) thereby indicating progress of treatment.

A calibration port 49 can be utilized to verify laser performance by placing the laser radiating device 14 in front of the port and operating the system 10. The port 49 determines whether the system 10 is operating within calibration specifications and automatically adjusts the system parameters. In at least one example, the port 49 may be external to the laser radiating device 14. In some examples, the laser radiating device 14 may have internal calibration components.

FIG. 2C is a block diagram of an exemplary control unit 12. Control unit 12 is configured to perform processing of data and communicate with laser radiating devices, for example laser radiating device 14 as illustrated in FIG. 2A. In operation, control unit 12 communicates with one or more of the above-discussed components and may also be configured to communication with remote devices/systems.

As shown, control unit 12 includes hardware and software components such as network interfaces 1210, at least one processor 1220, sensors 1260 and a memory 1240 interconnected by a system bus 1250. Network interface(s) 1210 can include mechanical, electrical, and signaling circuitry for communicating data over communication links, which may include wired or wireless communication links. Network interfaces 1210 are configured to transmit and/or receive data using a variety of different communication protocols, as will be understood by those skilled in the art.

Processor 1220 represents a digital signal processor (e.g., a microprocessor, a microcontrol unit, or a fixed-logic processor, etc.) configured to execute instructions or logic to perform tasks. Processor 1220 may include a general purpose processor, special-purpose processor (where software instructions are incorporated into the processor), a state machine, application specific integrated circuit (ASIC), a programmable gate array (PGA) including a field PGA, an individual component, a distributed group of processors, and the like. Processor 1220 typically operates in conjunction with shared or dedicated hardware, including but not limited to, hardware capable of executing software and hardware. For example, processor 1220 may include elements or logic adapted to execute software programs and manipulate data structures 1245, which may reside in memory 1240.

Sensors 1260 typically operate in conjunction with processor 1220 to perform measurements, and can include special-purpose processors, detectors, transmitters, receivers, and the like. In this fashion, sensors 1260 may include hardware/software for generating, transmitting, receiving, detection, and/or logging blood flow, hair follicles, and/or other parameters.

Memory 1240 comprises a plurality of storage locations that are addressable by processor 1220 for storing software programs and data structures 1245 associated with the embodiments described herein. An operating system 1242, portions of which may be typically resident in memory 1240 and executed by processor 1220, functionally organizes the device by, inter alia, invoking operations in support of software processes and/or services 1244 executing on control unit 12. These software processes and/or services 1244 may perform processing of data and communication with control unit 12, as described herein. Note that while process/service 244 is shown in centralized memory 1240, some examples provide for these processes/services to be operated in a distributed computing network.

It will be apparent to those skilled in the art that other processor and memory types, including various computer-readable media, may be used to store and execute program instructions pertaining to the fluidic channel evaluation techniques described herein. Also, while the description illustrates various processes, it is expressly contemplated that various processes may be embodied as modules having portions of the process/service 1244 encoded thereon. In this fashion, the program modules may be encoded in one or more tangible computer readable storage media for execution, such as with fixed logic or programmable logic (e.g., software/computer instructions executed by a processor, and any processor may be a programmable processor, programmable digital logic such as field programmable gate arrays or an ASIC that comprises fixed digital logic. In general, any process logic may be embodied in processor 220 or computer readable medium encoded with instructions for execution by processor 1220 that, when executed by the processor, are operable to cause the processor to perform the functions described herein.

FIGS. 3-19 illustrate different examples of laser radiating devices and different configurations and/or components that can be used in, on, and/or with laser radiating devices. While elements may be described with one example, the elements described herein may be utilized in any suitable example in any suitable combination.

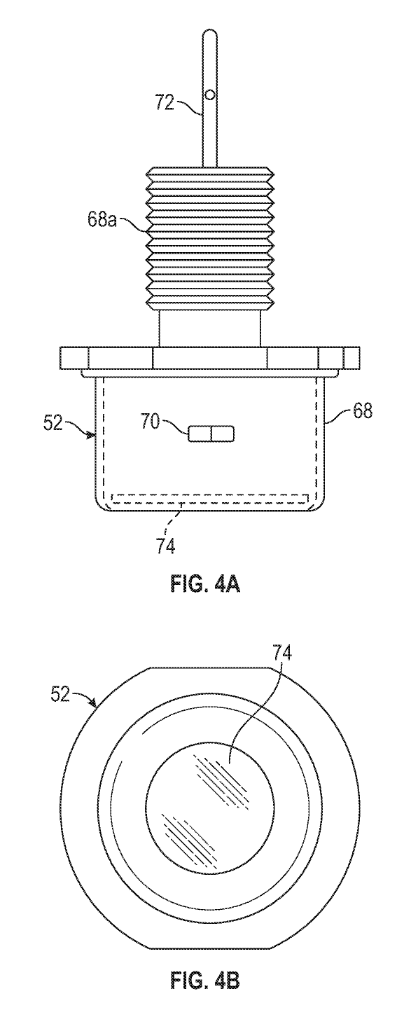

Referring to FIG. 3, the laser radiating device 14 can be sized to be easily manipulated by the user, for example by being hand held. In at least one example, the laser radiating device 14 can include a heat-conductive, metal bar 50. The bar 50 can be hollow along its central axis and is threaded on its interior at a first end for receiving a laser resonator 52, described further below with reference to FIGS. 4A and 4B. In at least one example, wiring 51 can extend from the resonator 52 through the hollow axis of the bar 50 for connection to the coaxial cable 16 (FIG. 2A). In some examples, the bar 50 can be copper or steel and thus conducts electricity for providing a ground connection for the resonator 52 to the cable 16.

A sleeve 54 is placed over the bar 50 for purposes of electrical and thermal insulation. In at least one example, the sleeve 54 can be made of material including glass noryl. In other examples, the sleeve 54 can be made of any suitable electrically and/or thermally insulative material such as rubber, glass, or by creation of a vacuum. A screw 55 extending through the sleeve 54 can anchor the sleeve 54 to the bar 50. In other examples, the sleeve 54 can be coupled with the bar 50 by adhesive. As shown, the resonator 52 is recessed slightly within the sleeve 54. An impedance o-ring 56, which can be at least partially formed of a conductive metal, is press-fitted into the end of the sleeve 54 so that when the laser radiating device 14 makes contact with tissue, the ring 56 touches the tissue. The ring 56 is electrically connected through the laser radiating device 14 to the unit 12. The ring 56 measures impedance by measuring angular DC resistance with an insulator ohmmeter, for example, of the tissue being irradiated by the laser radiating device 14 which is then displayed as impedance on the display 42. Any other suitable impedance measurement circuit may be utilized, as will be apparent to one skilled in the art.

A feedback sensor 57 can be located in the end of the sleeve 54 for measuring the output of the resonator 52. While not shown, the sensor 57 is connected electronically to the control unit 12 and to a feedback circuit within the control unit. A small percentage of the diode laser light from the resonator 52 is thus detected by the sensor 57 and channeled into the feedback circuit of the control unit 12 to measure and control performance of the resonator. Out-of-specification temperature, power, pulse frequency or duration is thus corrected or the system 10 is automatically turned off.

In at least one example, the laser radiating device 14 can include one or more sensors which are operable to measure one or more hair growth parameters. For example, the laser radiating device 14 can include a hair follicle sensor 11 and/or a blood flow sensor 13. The hair follicle sensor 11 can measure the number and/or the health of hair follicles. The blood flow sensor 13 can measure the amount of blood flow within a tissue of the subject. As such, the hair growth parameters can include number of hair follicles and/or blood flow, as well as any other suitable parameters which indicate hair growth and/or hair density. Based on the measured hair growth parameters, the laser radiating device 14 can determine one or more regions within boundaries to be treated. As such, the treatment can be focused only on the regions that need treatment instead of the entire subject.

For example, when the number of hair follicles is less than a predetermined number and/or the blood flow is less than a predetermined amount, it can be determined that the region needs to be treated. Additionally, the progression of the treatment can be determined by measuring the number of hair follicles and/or blood flow before treatment and following up with measurements of hair follicles and/or blood flow during and/or after treatment. Areas around the regions to be treated may acceptable hair growth parameters and may not need treatment.

As illustrated in FIG. 3, multiple metallic fins 58 can be placed over the end of the bar 50 and are separated and held in place by spacers 60 press-fitted over the bar 50. The fins 58 act as a heat sink to absorb heat from the laser through the bar 50 and dissipate it into the surrounding air. The spacers 60 placed between each fin 58 enable air to flow between the fins, thereby providing for increased heat transfer from the laser radiating device 14.

A casing 62 can be fit over the sleeve 54 and serves as a hand grip and structure to support a switch 64 and light 66. The switch 64 is used to actuate the laser radiating device 14 by the operator wherein the switch must be depressed for the wand to operate. The switch 64 is wired in a suitable manner to the control unit 12 and is used either alone or in conjunction with the foot pedal 22. The light 66 is illuminated when the laser radiating device 14 is in operation.

As shown in FIG. 4A, the laser resonator 52 can include a housing 68 having threads 68a configured for matingly engaging the threaded portion of the bar 50 in its first end. In other examples, other suitable coupling mechanisms can be utilized. A diode 70 is centrally positioned in the housing 68 facing in a direction outwardly from the housing 68. In at least one example, the diode 70 can be an Indium-doped Gallium Arsenide (In:GaAs) semiconductor diode. The diode 70 is electrically coupled with a power source, for example power supply 18 as illustrated in FIG. 2A. In at least one example, as illustrated in FIG. 4A, the diode 70 can be electrically connected to receive electric current through the threads 68a and an electrode 72 connected to the wiring 51 that extends longitudinally through the hollow interior of the tube 50 (FIG. 3). The amount of Indium with which the Gallium Arsenide is doped in the diode 70 can be an amount appropriate so that the diode 70, when electrically activated, generates and radiates, in the direction outwardly from the housing 68, optical energy such as low level reactive laser light having, at a power output level of 100-1000 mW. The diode 70 can radiate a predetermined wavelength ranging, for example, from about 1000 nanometers (nm) to 10,000 nm in the near-infrared region of the electromagnetic spectrum. Other types of diode semiconductor lasers may also be used to produce the predetermined wavelengths, e.g., Helium Neon, GaAs or the like.

As shown in FIGS. 4A and 4B, a lens 74 is positioned at one end of the housing 68 in the path of the generated laser light for focusing the light onto tissue treatment areas of, for example, 0.5 mm2 to 2 mm2, and to produce in the treatment areas an energy density in the range of from about 0.01 to about 0.15 joules/mm2. The lens 74 may be adjusted to determine depth and area of absorption.

The operating characteristics of the diode 70 are an output power level of 100-1000 mw, a center fundamental wavelength of 1000 nm to 10,000 nm, with a spectral width of about 5 nm, a forward current of about 1500 milliamps, and a forward voltage of about 5 volts at the maximum current.

In at least one example, the laser radiating device 14 can include diodes 70 having a wavelength of about 670 nm, activated at an undisclosed wattage. For example, the laser radiating device 14 can radiate wavelengths of from about 1,064 nm to about 2,500 nm for medical treatments that do not involve hair growth stimulation. Additionally or alternately, the laser diodes 70 can radiate optical energy at a predetermined wavelength within the region from about 2500 nm to about 10,000 nm can also be used for the stimulation of hair growth and tissue regeneration, and more specifically wavelengths in the region from about 2500 nm to about 5000 nm, and even more specifically wavelengths of about 3150 nm.

Broadly, the current disclosure includes systems, devices, and methods for a light source, typically a diode laser, operating in the infrared range at wavelengths of greater than about 1000 nm and, in some examples, at a low total wattage such as less than about 1,000 mw for the total output of the device, alternately less than about 500 mw. A laser operating in this range will have a greater dispersion rate than heretofore, thus requiring fewer diodes to cover the same area of scalp stimulation for promoting hair growth. A number of factors govern effective scalp stimulation: laser diode wavelength and power (diode wattage); light beam divergence and dispersion; duration period of laser light application/stimulation; rate of application, i.e. the number of periods per unit of time; and the distance between the diodes and the scalp. A minimal spacing may be more effective when using diodes in this infrared range and at low wattage.

In at least one example, the laser radiating device 14 can be used for purposes of appetite suppression key acupuncture/acupressure points which can be located on the ears, face, lower arm (forearm) and hands. The surface of the tissue in the region to be treated is irradiated with the laser beam light to produce the desired therapeutic effect. Because laser light is coherent, a variable energy density of the light of from about 0.01 to 0.15 joules/mm² is obtained as the light passes through the lens 74 and converges onto each of the small treatment areas. The energy of the optical radiation is controlled by the power control 30 and applied (for durations such as 1 minute to 3 minutes, continuous wattage or pulsed, for example) as determined by treatment protocols, to cause the amount of optical energy absorbed and converted to heat to be within a range bounded by a minimum absorption rate sufficient to elevate the average temperature of the irradiated tissue to a level which is above the basal body temperature, but which is less than the absorption rate at which tissue is converted into a collagenous substance. The laser beam wavelength, spot or beam size, power dissipation level, and time exposure are thus carefully controlled to produce in the irradiated tissue a noticeable warming effect, which is also limited to avoid damaging the tissue from thermal effects.

The present disclosure has several advantages. For example, by using an In:GaAs diode laser to generate the laser beam energy, the laser source can be made sufficiently small to fit within the hand-held laser radiating device 14, thereby obviating the need for a larger, more expensive laser source and the fiber optic cable necessary to carry the laser energy to the treatment tissue. The In:GaAs diode laser can also produce greater laser energy at a higher power dissipation level than lasers of comparable size. Furthermore, construction of the laser radiating device 14 including the fins 58 provides for the dissipation from the wand of the heat generated by the laser source. In addition, while the present example illustrated in FIG. 2A only includes one laser radiating device 14, it should be understood that multiple laser diodes and wands may be used to treat large patients or to treat multiple acupuncture/acupressure points simultaneously.

It is understood that several variations may be made in the foregoing without departing from the scope of the invention. For example, any number of fins 58 may be utilized as long they dissipate sufficient heat from the laser radiating device 14 so that the user may manipulate the wand without getting burned. The setting controls 24 may be used individually or in combination and the information displayed on the displays 26 may vary. Other diode laser structures may be utilized to produce the desired effects.

While the ring 56, feedback sensor 57, resonator 52, hair follicle sensor 11, and blood flow sensor 13 are illustrated in FIG. 3 as being at the end of the sleeve 54, in other examples, one or more of the ring 56, feedback sensor 57, resonator 52, hair follicle sensor 11, and blood flow sensor 13 can be disposed along a side of the laser radiating device 14 for ease of the user.

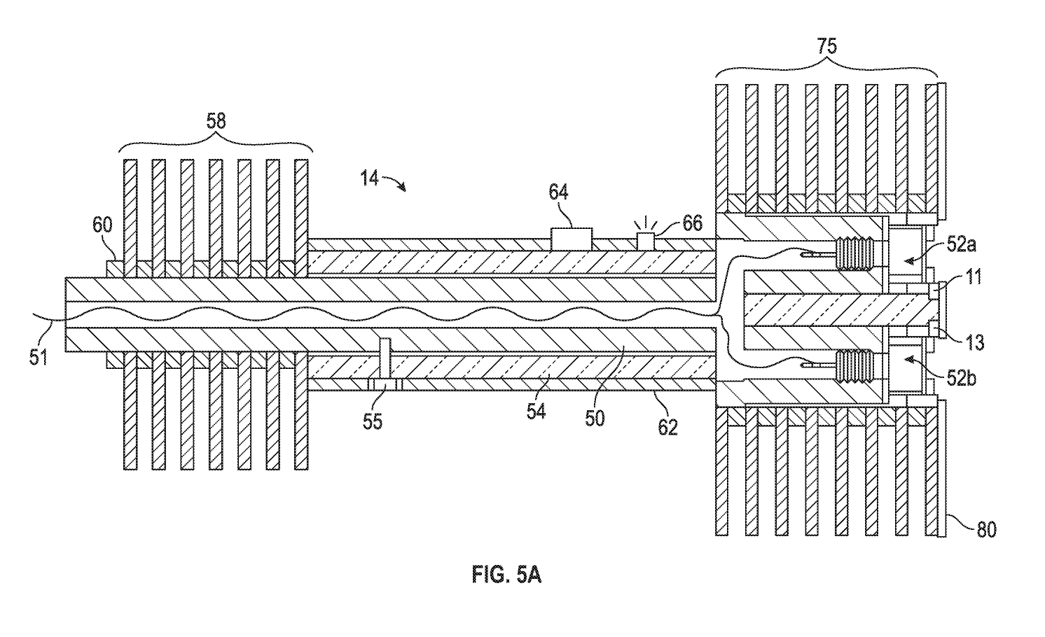

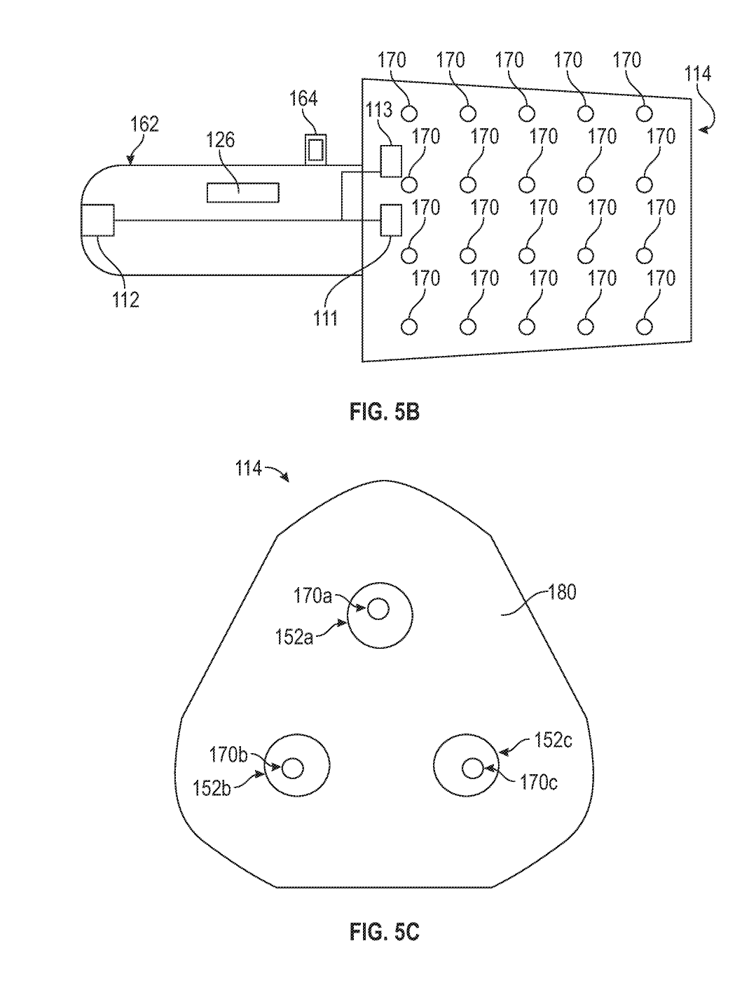

For example, as illustrated in FIGS. 5A-5C, an exemplary laser radiating device can include a plurality of light sources 170. As illustrated in FIG. 5A, the laser radiating device 14 can include more than one laser resonator 52a, 52b. As the laser radiating device 14 can include more than one laser resonator 52a, 52b, the size of the laser radiating device 14 may be increased. For example, the width of the laser radiating device 14 may be about 2.5 cm wide. Additionally, in some examples as illustrated in FIG. 5A, the laser radiating device 14 can include an additional set of metallic cooling fans 75 surrounding the laser resonators 52a, 52b at the terminal end of the laser radiating device 14. The additional cooling fins 75 can be used due to the increased heat produced by the plurality of light sources, for example the three light sources 170a-170c as illustrated in FIG. 5C. This increased heat is not only due to the increased number of diodes and resonators in the laser radiating device 14 but also the density of heat generated within a given volume. Accordingly, the additional metallic fins 75 surrounding the resonators 52a, 52b provide adequate local heat dissipation to prevent thermal damage to the tissue that cannot be achieved by relying solely on the fins 58 on the far end of the laser radiating device 14. In at least one example, a cover plate 80 can be included to provide additional thermal protection of the tissue. In some examples, the cover plate 80 can include a plastic material.

As illustrated in FIG. 5B, the laser radiating device 114 can include a plurality of light sources 170, a hair follicle sensor 111, and a blood flow sensor 113 disposed on a side of the laser radiating device 114. The laser radiating device 114, as illustrated in FIG. 5B, include 20 light sources 170 in an array of 4×5 light sources 170. The number and configuration of the light sources 170 is not limited to the illustrated examples. For example, the shape of the laser radiating device 114 is not limited to the rectangular shape as illustrated in FIG. 5B and can be ovoid, cylindrical, triangular (for example as illustrated in FIG. 5C), and/or irregular shaped. As illustrated in FIG. 5C, the laser radiating device 114 can include three light sources 170 forming a triangular shape. For example, ten diodes 170 may form a circular shape.

Additionally, as illustrated in FIG. 5B, the display 126, switch 164, and/or control unit 112 (similar to control unit 12) are disposed within the laser radiating device 114. For example, the display 126, switch, 164, and/or control unit 112 can be disposed within the handle 162 of the laser radiating device 114.

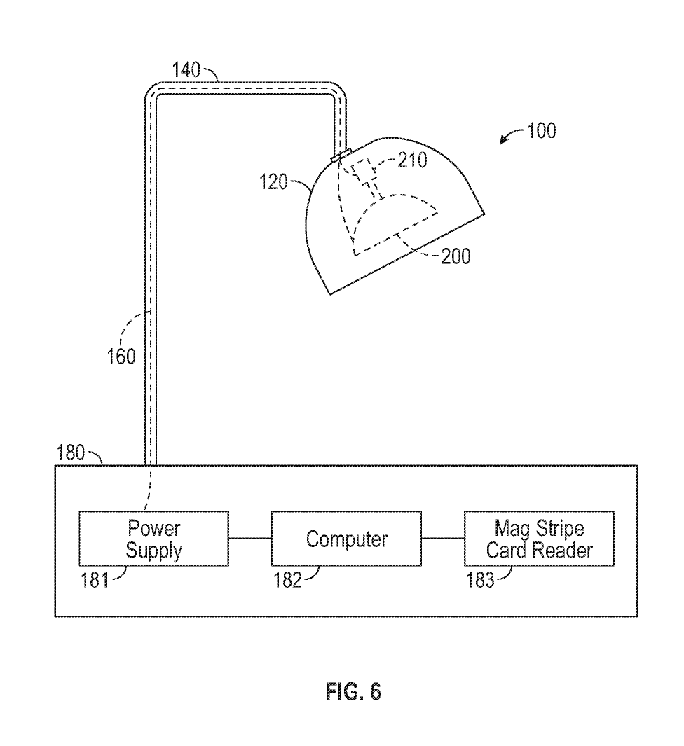

FIG. 6 depicts another example of the system 100, which includes a stationary cap 120 provided for surrounding and covering a subject's head, in a manner resembling a well-known hair dryer. In at least one example, the shape of the stationary cap 120 can be any other suitable shape such as having a lesser curvature, be rectangular, or any other suitable configuration.

The cap 120 may be supported on a cantilevered support 140 to allow the cap 120 to be positioned over and about the head of a subject while maintaining a non-contact spacing between the interior of the cap 120 and the scalp. As such, the subject may receive treatment without the need of any person holding the laser radiating device. The subject's head may optionally be supported by an external chair having a neck rest (not shown) so that spacing between the scalp and the interior of the cap 120 may be maintained. The cap 120 may provide stable support for a cap 200 therein, with the cap 200 being actuated for rotation by a motor 210.

A wiring harness 160 may be provided between the cap 120 and a control unit 180 that provides control and power to components contained within the cap 120. In the example shown, the wiring harness 160 may be routed through a hollow interior of the cantilevered support 140 for convenience and to protect the wiring harness 160 from snagging or damage. However, the wiring harness 160 may also be attached directly to the cap 120 by means of a coiled cable, a bundle of bound wires, or other means well known to the art.

The control unit 180, which can be similar to control unit 12 as shown in FIGS. 2A and 2C, may include a power supply 181, a computer 182, an optional magnetic stripe card reader 183, and/or manual controls (not shown). The power supply 181 may be of standard design having sufficient capacity to power a computer 182, actuate the motor 210 within the cap 120 and to drive light sources within the cap 200, as will be described presently. The computer 182 may provide control to the motor and light sources and receive direction from manual controls (not shown) associated with the control unit 180. The magnetic stripe card reader 183 may be representative of various input devices well known to the art, which allow data to be provided to and received by the computer 182.

It should be understood that the configuration described above is representative of the system 100 and obvious modifications providing the same functionality may be used within the scope of the invention. For example, in some examples, the wiring harness 160 may be replaced by a wireless protocol in which the control unit 180 may broadcast control information to a receiving unit located in the cap 120, with the control unit 180 and the cap 120 having their own independent power supplies 181. The magnetic stripe card reader 183 may be substituted with a flash memory card, a near-field communicator, or a floppy disk reader. Other obvious modifications may be contemplated as being within the scope of the invention.

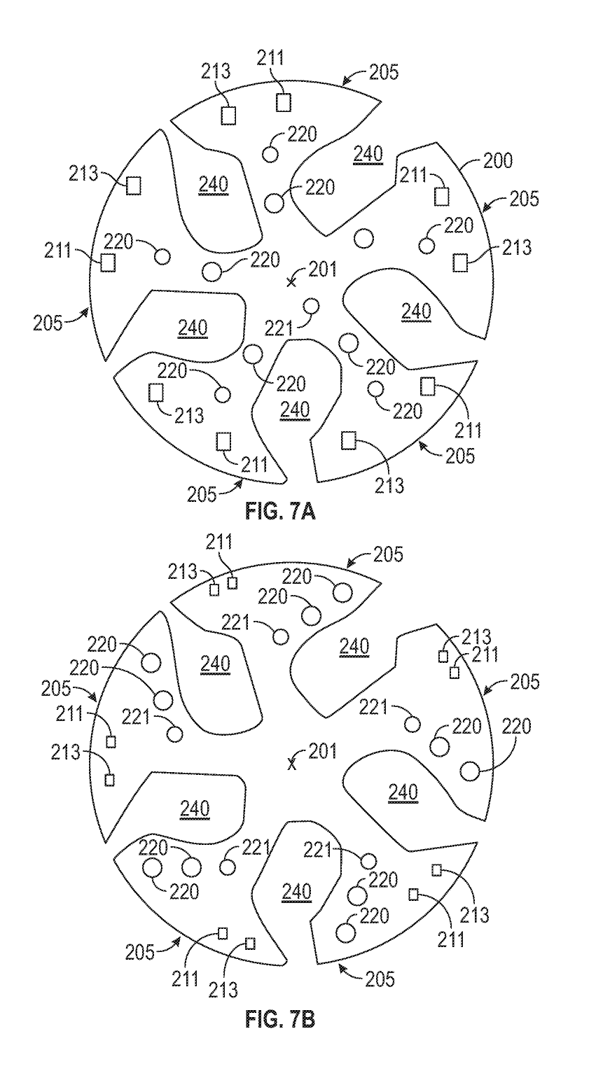

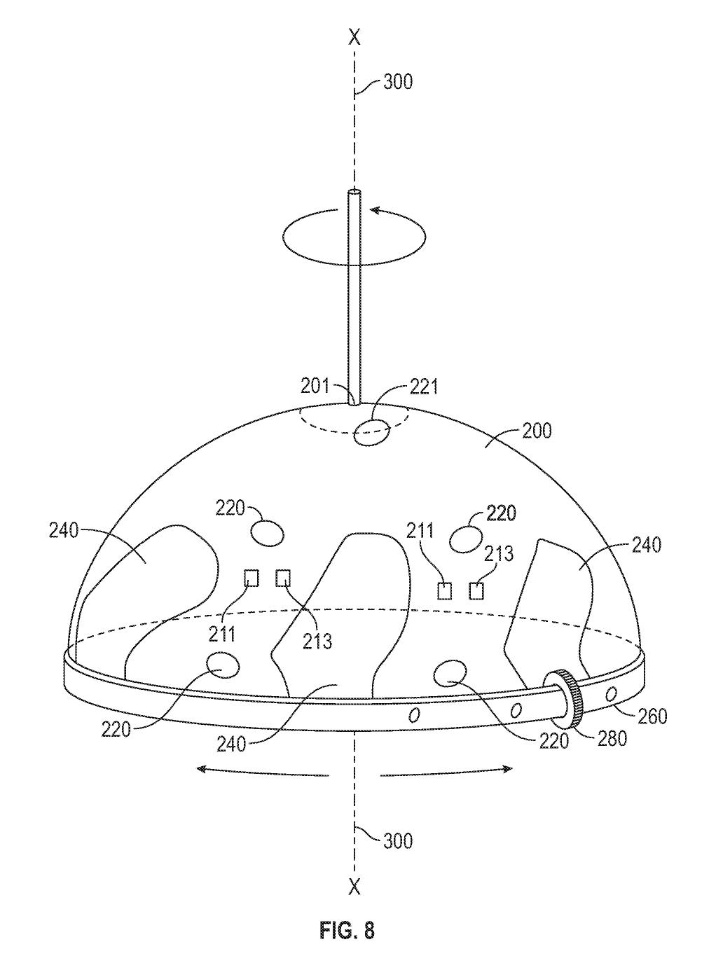

The cap 200 contained within the cap 120 may be of a generally circular aspect. An exemplary flattened pattern for the cap 200 is shown in FIGS. 7A and 7B, which has a center of rotation 201. Cutouts 240 may be removed from the flattened pattern to allow the resulting shape to assume a three-dimensional form as by bending or folding the portions, or modules 205, remaining between the cutouts 240. The cap 200 may be formed by folding each module inwardly in the same direction to form what geometrically is known as a spherical cap (FIG. 8), which is defined as the shape resulting from a plane passing through a sphere. The diodes 220 in the cap 200 may be inwardly directed towards the interior of the cap 200. In at least one example, the cap 200 may include one or more sensors, for example a hair follicle sensor 211 and/or a blood flow sensor 213, to measure hair growth parameters as described above. In at least one example, each of the modules 205 can include a hair follicle sensor 211 and/or a blood flow sensor 213 to measure the hair growth parameters at regions that correspond with the diodes 220, 221.

The cap 200 formed may be sized to allow its shape to be fitted over and around the patient's head for rotational movement without making firm contact with the patient's head. The cap may extend so far as to form a geometric hemisphere. In at least one example, the spherical cap forming cap 200 may form from one-half to one-third of a hemisphere. Cap 200 may be fabricated of a thin, durable flexible material, which can be formed into the spherical cap shape as shown in FIG. 6.

Referring to FIG. 8, an adjustment strap 260 may be provided about the cap 200, for example at the bottom of the cap 200. The adjustment strap 260 may include an adjustment knob 280 to adjust the shape of cap 200 to accommodate various head sizes. In other examples, the adjustment strap 260 may be overlapped and secured by using a standard hook-and-loop device. Other devices for adjusting and securing the strap to accommodate differing head sizes may be used without departing from the scope of the invention.

Cap 200 may be designed for rotation about an axis 300 that passes through the center of rotation 201. Such rotation may be accomplished through any conventional motor means known to the art. The number of diodes 220, the placement of the diodes 220 about the cap 200, the cyclical sequence of rotational movement, and the actuation of the diodes 220 may be design choices that depend upon the areas of the scalp that are intended to be stimulated for hair growth.

In the example shown in FIGS. 7A, 7B, and 8, five pairs of circumferentially-spaced diodes 220 may be placed in modules 205 so that they flank cutouts 240 in cap 200. An eleventh diode 221 may be located near center of rotation 201. Although only 11 diodes 220, 221 are shown for illustrative purposes, as many as 20 to 30 single diodes 220 may be placed in cap 200 so that they traverse the area of interest on the scalp. Additionally and without departing from the scope of the invention, the site for each diode 220 may include a cluster of diodes 220, so that the area traversed by the cluster is broader than the area traversed by a single diode 220. It should also be noted that the spacing of diodes 220, 221, as shown in FIGS. 7A, 7B, and 8, is not to scale and is understood to be for illustration purposes only.

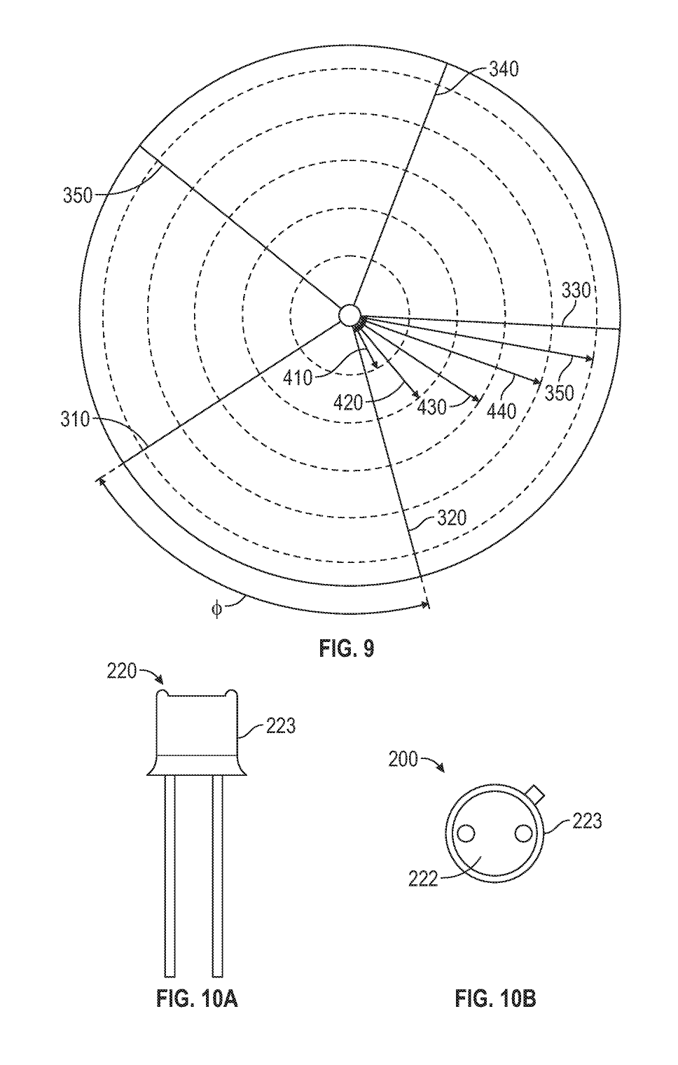

Referring now to FIG. 9, a polar view is presented of cap 200, showing an exemplary schema for describing different patterns for the placement of light sources 220. Here, a plurality of rays 310, 320, 330, 340, 350 are shown, along which light sources 220 may be positioned according to radii 410, 420, 430, 440, 450. According to the example using five pairs of light sources 220, five rays 310-350 may be postulated, each ray being spaced equidistantly according to angle φ. As a practical matter, each ray 310-350 may fall upon the cap material that remains between two cutouts 240. Two light sources 220 may be positioned at radii 410-450 along a ray 310-350, so that a selected portion of the scalp is traversed. Alternatively, light source 221 may be eliminated. Other spacing patterns may be used according to patterns well known to those skilled in the art.

It is to be understood that the present device may accommodate multiple caps 200, each cap being replaceable by another cap 200 having a different light source arrangement therein. The wiring harness 160 may have a standard coupling arrangement for a maximum number of light sources accommodated by the device, so that each light source in the cap 200 is associated with a particular “address” or number. In this way, alternative light source arrangements may be controlled in a known and established manner, according to how the control unit 180 is programmed. Furthermore, each cap 200 may be equipped with a standard universal mount well known to the art, e.g. a bayonet arrangement, which permits the cap to be removably attached to the bonnet 120 and motor 210, so that caps 200 may be exchanged as the need arises.

The control unit 180 may be adapted to accepted parameters selected by the operator, such as speed of rotation of the cap, angle of rotation, direction (clockwise or counterclockwise), and actuation of the diodes (i.e., points of time at which a particular diode 220 may be turned on or off). A group of such parameters may determine a cyclical sequence that may be stored in the control unit 180 for convenience. A cyclical sequence may be developed for different patterns of hair loss, stored within the control unit 180, and retrieved as needed, depending upon the patient. For example, in at least one example, the cap 200 may be rotated in one direction intermittently in increments of 36° for periods of 60 sec. each period, so that diode 221 treats the entire of the top of the scalp. If two diodes 221 were provided with a spacing of 180° apart, then a cycle pattern having only 180° of rotation might be required. This rotation may be performed in the same direction for as long as treatment is programmed, or it may be reversed every 180° or 360°, depending on the options that are made available to the operator, which can take many forms, as will readily occur to one skilled in the art. Accordingly, the region to be treated can be focused on by moving at least one of the plurality of modules 205 to position at least one of the modules 205 above the region to be treated. The diodes 220, 221 disposed on the at least one module 205 positioned above the region to be treated can radiate optical energy at the predetermined wavelength, and the optical energy is radiated within the boundaries of the region. As such, only the region(s) to be treated are focused on.

In other examples, the cap 200 may not be rotated, and only the diodes 220, 221 positioned above the region(s) to be treated are activated to radiate optical energy. For example, the control unit 180 may be programmed to actuate individual light sources at different power levels and the cap 200 held stationary. Each individual light source may be programmed to illuminate an area of the scalp for a given amount of time and then to cease operation for a given amount of time, with the cyclical sequence thus defined to be repeated for a specified number of repetitions. In this way, areas of the scalp exhibiting severe hair loss may be treated with coherent light at slightly higher power levels simultaneously with other areas that may exhibit only moderate hair loss. In yet other examples, the diodes 220, 221 may radiate optical energy which is then directed to the region to be treated, for example by fiber optic cables.

An example of a light source 220 that may be used according to the present disclosure is shown in detail in FIGS. 10A and 10B. The light source 220 shown may be of a standard construction and design, with a window at the top for emitting coherent light, such as by a laser. For example, the light source 220 may be a Boston Electronics Model LED34-05 diode, having a window cap that is 3.5 mm in diameter (approx. 0.15 in.). This diode has a peak emission wavelength of 3400 nm (3.4 microns) and a maximum emissive power of 20 pw at 2.5% duty cycle in pulsed mode. Diodes of this type may also be operated in continuous mode without departing from the scope of the present disclosure.

The light sources 220 may radiate optical energy at predetermined wavelengths. For example, the wavelengths may be between about 800 nm and about 10,000 nm. In some examples, the predetermined wavelength can be between about 1300 and about 4000 nm. In some examples, the predetermined wavelength can be between about 1550 nm and about 3150 nm.

Light sources of this type may operate at a power level, for example individually about 100 mw. In some examples, the power level of each of the light sources can be about 500 mw. In some examples, the power level of each of the light sources can be between about 100 mw and about 500 mw. In some examples, the power level of each of the light sources can be greater than 100 mw. The collective power level of the laser radiating device is then dependent on the number of light sources. For example, if each light source has a power level of 100 mw and there are 3 light sources, then the collective power level of the laser radiating device is at least 300 mw. The beam divergence/dispersion of this diode may be controlled by means of a lens 222 in the top of the cap 223 surrounding the diode. The lens 222 will exhibit the narrowest dispersion, while a diode cap 223 having no lens will exhibit intermediate dispersion and a capless diode will exhibit the widest dispersion. The divergence/dispersion pattern chosen may be dependent upon the distance between the surface of the scalp and the light source 220, so that sufficient coverage of region to be treated may be achieved.

The light sources described herein for stimulating hair growth may typically be operated at a collective power level depending on the application and/or the number of light sources. For example, in the case of cancer patients, the chemotherapy used to treat the cancer will frequently result in severe hair loss. Such patients have been found to require higher levels of hair follicle stimulation than the normal patient population. These higher levels of stimulation may be, for example, provided by power levels that exceed 500 mw for the collective laser light sources but generally not exceeding 2700 mw collectively. Power levels lower than 500 mw and greater than 2700 may be utilized without deviating from the scope of the inventive concept.

The apparatus thus described may be used to promote hair growth from tissue of a subject according to a method of the present disclosure. According to the method, one or more of the light sources 220, 221 may be arranged along the inner surface of the cap 200 according to a fixed pattern. A periodic cycle may be programmed into the control unit 180 that actuates the cap 200 and light sources 220, which will cause the cap 200 to move in a repeated periodic movement about the scalp. The cap 200 may be arranged so that each light source 220 in the cap 200 is at the same general distance from the scalp. The cap 200 may then be allowed to periodically cycle through its programmed course for a fixed length of time. Multiple treatments of this type may be necessary to complete the hair stimulation process.

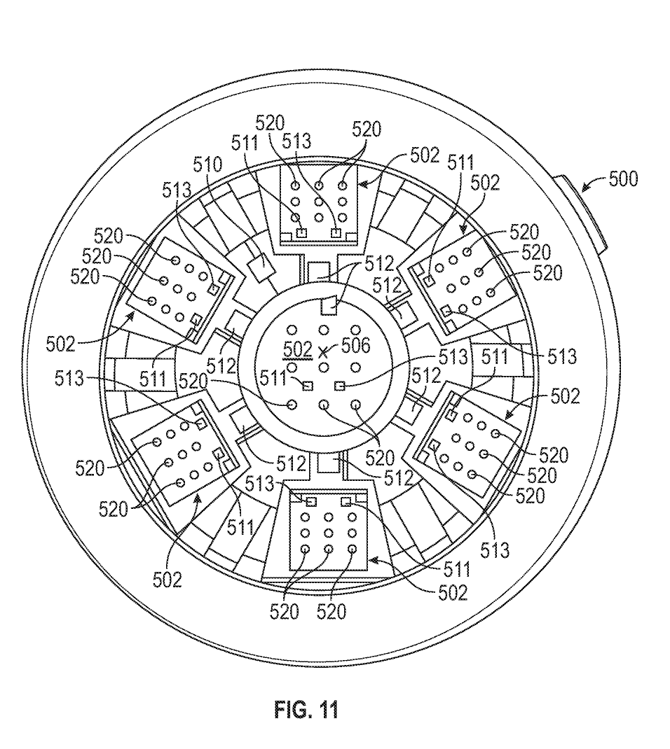

FIG. 11 shows an alternative exemplary system 500 in which each laser module 502 has its own control unit 512 (similar to control unit 12). Each of the modules 502 includes at least one light source 520 and/or at least one sensor 511, 513 to measure hair growth parameters, for example as discussed above. The sensors 511, 513 can include a hair follicle sensor 511 and/or a blood flow sensor 513. This example represents a distributed (modular) design. Instead of one centralized control board to control all of the diodes, the exemplary system 500 shown in FIG. 11 has seven separate control units 512. In at least one example, seven control units 512 are used to control each of the seven module 502. The number and configurations of the modules 502 is not limited to the illustrated example. For example, one, two, or more modules 502 can be included. In some examples, the modules 502 may be arranged in an array instead of arranged about a central point 501. Each of the modules 502 has its own control unit 512 including a processor coupled with the light sources 520 and/or at least one of the sensors 511, 512 and a memory. By changing to the distributed (modular) model, the exemplary system minimizes the number of wires to make the clinical unit much more robust. With this approach, power can be brought to each module 502 individually. Additionally or alternately, each module 502 can be moved as desired. For example, all modules 502 can be rotated together. In other examples, individual modules 502 can be moved without moving other modules 502. As such, the regions to be treated can be focused upon.

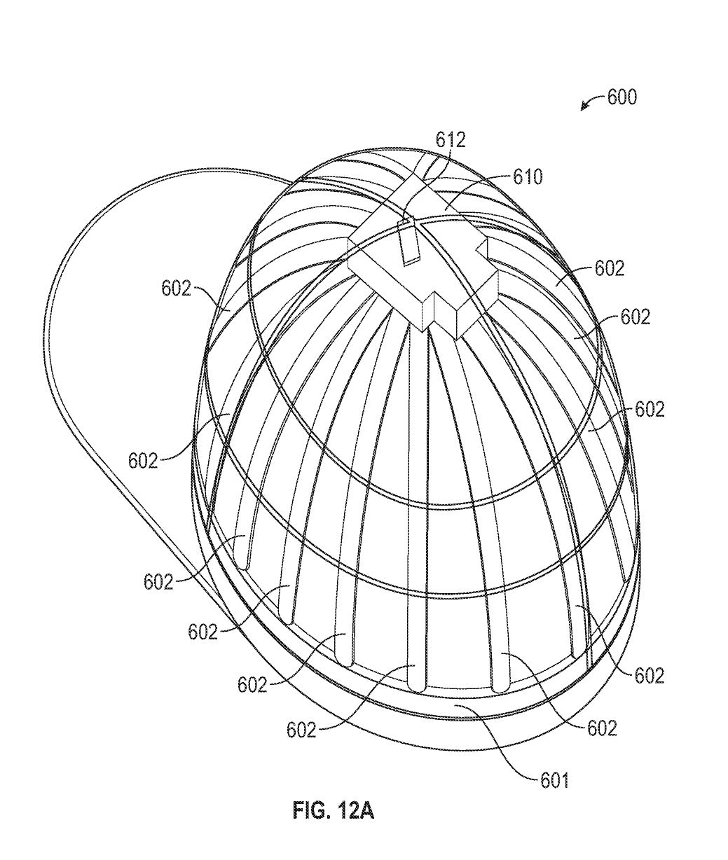

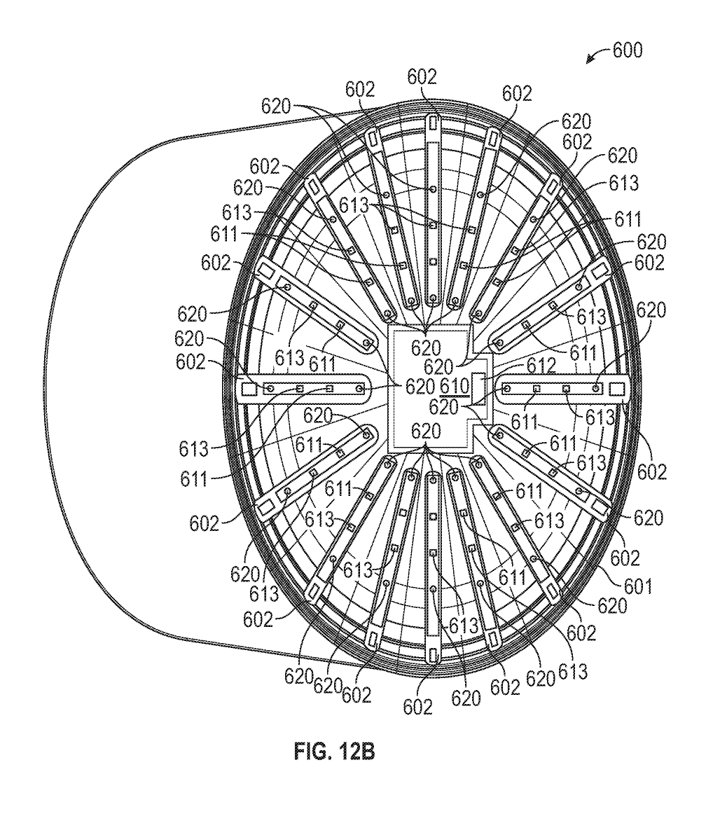

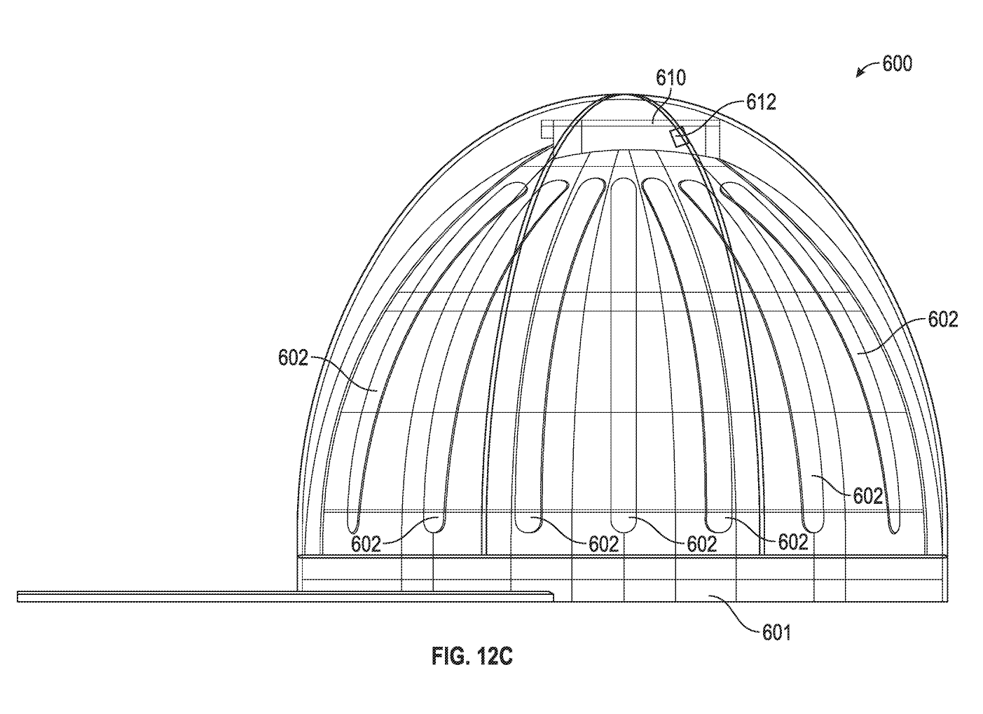

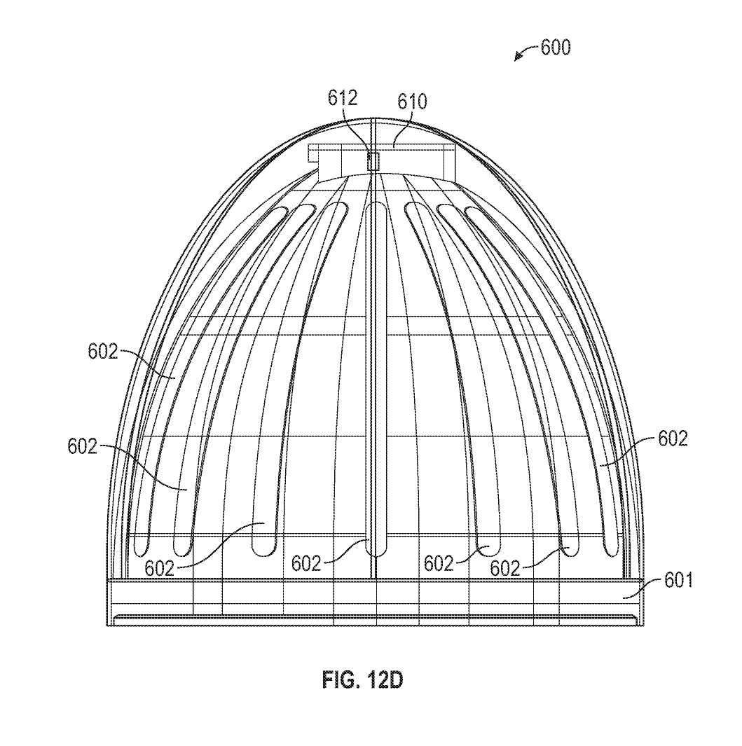

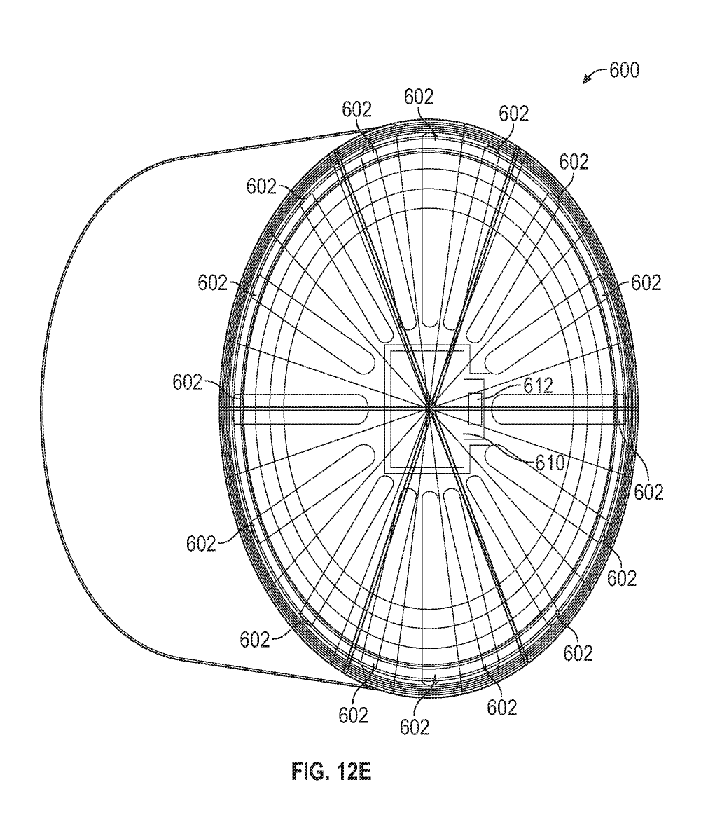

FIGS. 12A-12E show an interchangeable modular laser diode cap 600 in accordance with an example of the system. The Interchangeable/Modular cap 600 can provide both Clinical and Home version with a modular concept. The cap 600 can be interchangeable. For example, the cap 600 can be a soft fabric hat, such as one used at home, and can also be a dome such as one used in a clinical setting. The cap 600 includes a frame 601. Modules 602 are operable to be coupled with the frame 601. For example, the frame 601 can include snap base plates to allow the modules 602 to couple with the frame 601. In at least one example, the frame 601 can include a built in mechanism to permit the modules 602 from removably coupling with the frame 601. The built in mechanism can be, for example, hook and loop fasteners, snap fit fasteners, and/or adhesive. In at least one example, the cap 600 may include a motor 610 which can move the modules 602 to focus the treatment on the regions to be treated.

The modules 602 can include a band of light sources 620, a cluster of light sources 620, and/or a matrix of light sources 620 such as 5×5 or 6×6. The light sources 620 can radiate optical energy at predetermined wavelengths to treat one or more regions of a subject to promote hair growth as discussed above. If two or more light sources 620 are configured, then the distances between adjacent light sources 620 may be equal to each other or the distances between any pair of adjacent light sources 620 may be different from the distance between any other pair of adjacent light sources 620, without departing from the scope of the invention. The light sources 620 configured within the cap 600 may provide near infrared radiation having a wavelength that is with a region from about 100 nm to about 10,000 nm, alternately within a region from about 1300 nm to about 4000 nm, and alternately from about 1550 nm.

Each light source 620 may be operated at a power level of about 100 mw, alternately greater than 100 mw. In at least one example, the power level of each light source 620 can be less than 100 mw. The power level applied to each light source 620 may be independently controlled without affecting the power level applied to other light source 620, without departing from the scope of the invention. Each module 602 within the cap 600 may have a spacing between light sources 620 that differs from the spacing for other modules 602, in order to provide more complete coverage of the regions to be treated. The movable modules 602 may be configured with a constant angular displacement from an adjacent movable band, with all modules 620 moving as a unit. In some examples, each module 602 may be independently movable.

The cap 600 can include sensors 611, 613, for example a hair follicle sensor 611 and/or a blood flow sensor 613 as discussed above to measure hair growth parameters. In at least one example, each of the modules 602 can include sensors 611, 613.

In at least one example, the cap 600 includes a control unit 612 coupled with the sensors 611, 613 and the light sources 620, similar to control unit 12. Based on the hair growth measurements by the sensors 611, 613, the control unit 612 can control the light sources 620 and/or the modules 602 to focus treatment on regions that need treatment. Additionally, the control unit 612 can adjust the wavelengths and/or the intensity as needed to optimize hair growth. In other examples, each of the modules 602 can include a control unit 612. As such, the modules 602 can be interchangeable, and the cap 600 can be modular. For example, different shapes of modules 602 and different wavelengths and different power levels can co-exist in the same system.

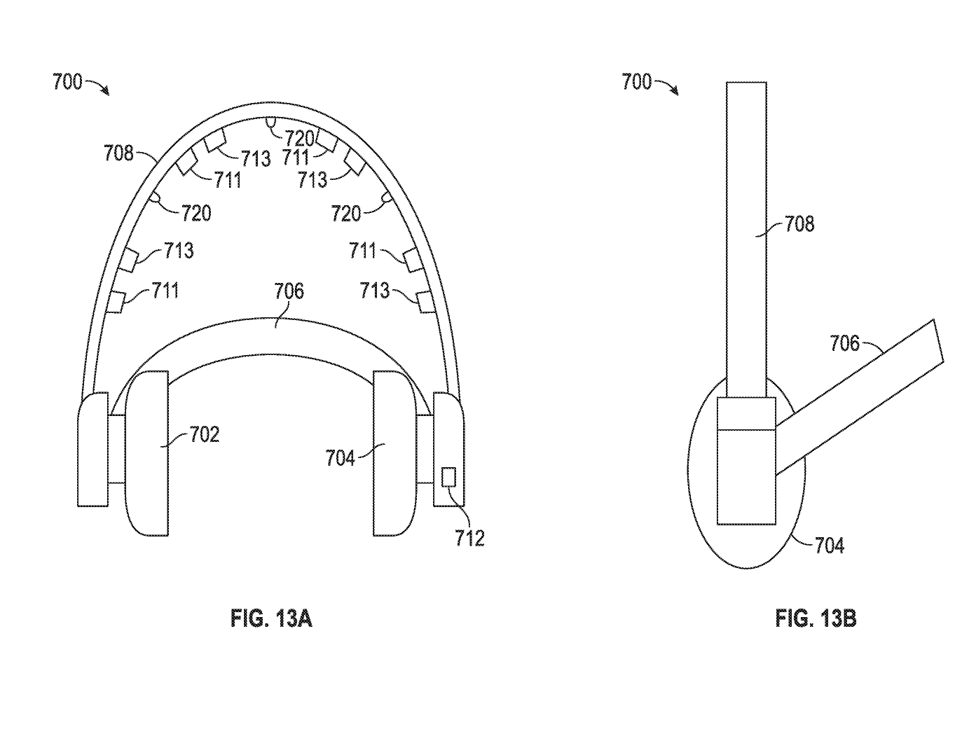

Referring now to FIGS. 13A and 13B, a laser radiating device 700 is shown as a hair band stimulation device, according to an example of the present disclosure. A pair of ear cups 702, 704 may be fixedly positioned over the ears and maintained at a constant angular displacement about the head by a stabilizer 706. One or both of ear cups 702, 704 may include a motor (not shown) that moves a movable band 708 over the scalp in a controlled manner.

The movable band 708 may contain one or more light sources 720 along its inner surface, each light source 710 being positioned to shine in a direction that is more or less perpendicular to the scalp surface. In certain examples, the distance between light sources 720 and the subject's scalp may be maintained at all points within a known tolerance range. If two or more light sources 720 are configured, then the distances between adjacent sources 720 may be equal to each other or the distances between any pair of adjacent sources 720 may be different from the distance between any other pair of adjacent sources 720, without departing from the scope of the present disclosure. The light sources 620 configured within the cap 600 may provide near infrared radiation having a wavelength that is with a region from about 100 nm to about 10,000 nm, alternately within a region from about 1300 nm to about 4000 nm, and alternately from about 1550 nm.

Those of skill in the art will understand and appreciate that the above specific frequency ranges are provided only by way of example, and that light sources able to emit light anywhere within the range between approximately 1000 nm and approximately 10,000 nm may be employed in certain examples of the present disclosure. It is possible that frequencies below 1000 nm may be employed in certain examples. It is also possible that frequencies above 10,000 nm may be employed in certain examples. Certain examples may employ two or more light frequencies, which may be within or outside of the above-referenced frequency ranges.

In at least one example, the movable band 708 can include sensors 711, 713, for example a hair follicle sensor 711 and/or a blood flow sensor 713, to measure hair growth parameters as described above.

Movable band 708 may be pivotally moved over the surface of the scalp within a certain range. As an example, movable band 708 may rotate over a region of the scalp from about the nape of the neck to about the forehead of the subject. By controlling the extent of travel of movable band 708 over the scalp surface, the power intensity of the light sources 720, and the on/off status of the light sources 720, different areas of the scalp may be targeted for radiation while leaving other areas of the scalp alone.

As shown in FIGS. 13A and 13B, the stabilizer 706 may be a solid band about the back of the head that compressively maintains ear cups 702, 704 over the ears without rotating ear cups 702, 704. The stabilizer 706 may additionally include supports (not shown) and other devices that will position ear cups 702, 704 against the shoulder and other body parts. The stabilizer 706 may thus provide a fixed frame of reference within which an angular rotation of the band may take place. The stabilizer 706 shown in FIGS. 13A and 13B may be exemplary and should not be taken as limiting the present disclosure to the example shown in FIGS. 13A and 13B.

Each of ear cups 702, 704 may contain a motor for moving the movable band 708 over the scalp. In certain examples, a single motor may be used on one of ear cups 702, 704 with the other ear cup providing a rotational bearing facilitating angular movement of the movable band 708, without departing from the inventive concept. Either or both of ear cups 702, 704 may also contain electronic means for providing music, radio, instructions to the patient, and other audio sources to the patient's ears in order to entertain the patient during the radiation process. The ear cups 702, 704 may also have a soft cushion to prevent discomfort during the radiation process.

Although FIGS. 13A and 13B show a single movable band 708, multiple bands may be configured for angular movement over the scalp around ear cups 702, 704. Each movable band 708 may have a spacing between light sources 710 that differs from the spacing for other bands 708, in order to provide more complete coverage of the scalp. The movable bands 708 may be configured with a constant angular displacement from an adjacent movable band 708, with all bands moving as a unit. In other examples, each movable band 708 may be moved independently from one another.

A control unit 712 (similar to control unit 12) attached to the device 700 may be adapted to accepted parameters selected by the operator, such as the speed of movement of the band 708, the angle of rotation, direction (forward or back), actuation of the light sources 720 (i.e., points of time at which a particular light sources 720 may be turned on or off) and power level of each light sources 720 on each band 708. This set of parameters may be termed a cyclical sequence. The cyclical sequence may be stored in the control unit 712 for convenience. A cyclical sequence may be developed for different patterns of hair loss, stored within the control unit 712, and retrieved as needed, depending upon the subject.

In certain examples, a periodic cycle may be programmed into the control unit 712 that actuates movable band 708 and light sources 720, which will cause movable band 708 to move in a repeated periodic movement over the scalp. The movable band 708 may then be allowed to periodically cycle through its programmed course for a fixed length of time. Multiple treatments of this type may be necessary to complete the hair growth stimulation process.

Although the principal example described herein may employ laser diodes as an example light source, there is nothing within the spirit and scope of the present disclosure limiting the light sources to laser diodes, specifically. Depending on the specific application, light may be generated via a variety of laser types, including gas lasers, chemical lasers, dye lasers, metal-vapor lasers, solid-state lasers or semiconductor lasers. It is not necessary that the light used in the present disclosure be generated by a laser. A variety of suitable light sources may be employed in the present disclosure, as will be known to, and appreciated by, those of skill in the art. Further, any suitable devices capable of generating, shifting, refracting, reflecting, polarizing, diverting, focusing or filtering light in such a manner as to provide light at the correct location within the proper frequencies and at the proper level of intensity may be used to generate and direct light in connection with the examples disclosed herein. These devices may include, but are not limited to, fiber optics, conduits, mirrors, lenses, prisms and filters.

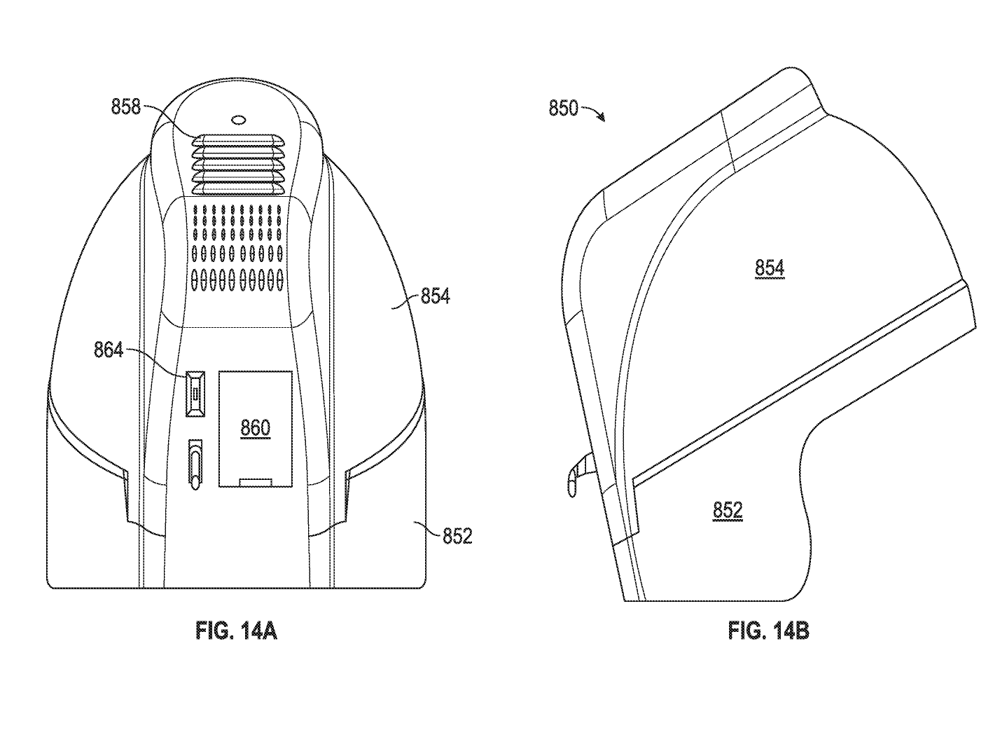

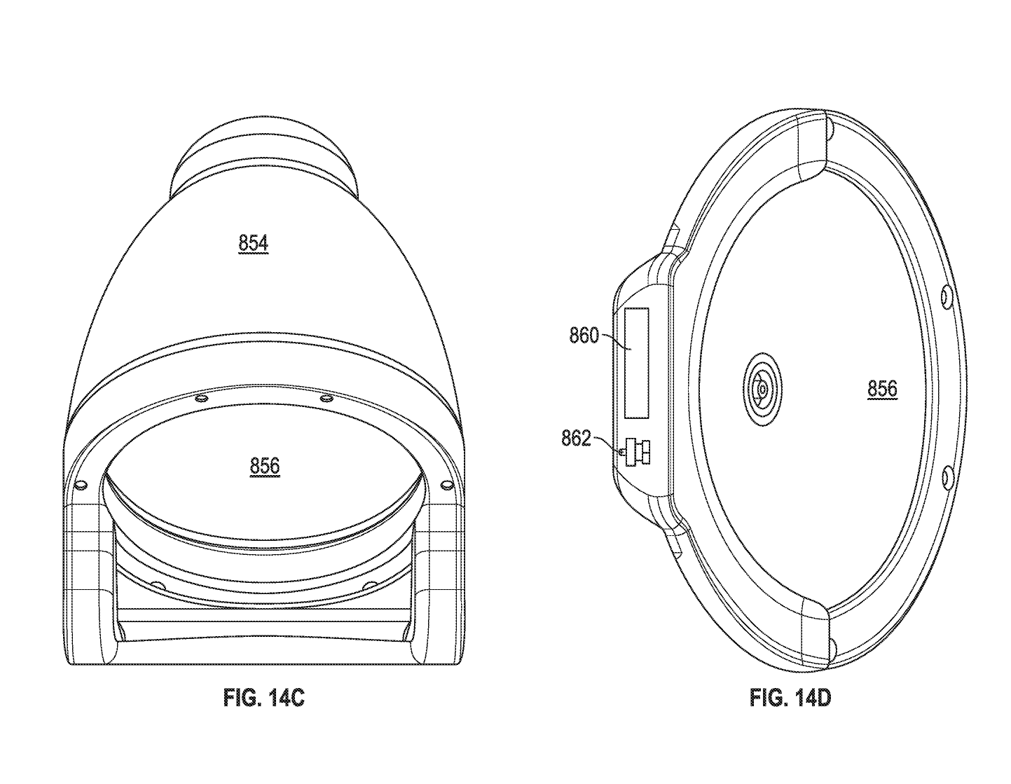

FIGS. 14A-14D show rear, side, front and bottom views, respectively, of a laser radiating device 850, for example a light application helmet according to one example of the present disclosure. The laser radiating device 850 includes a frame 852, an upper housing 854, and an inner dome 856. Frame 852 provides structural support for the various components of laser radiating device 850. Upper housing 854 protects the internal components of light application laser radiating device 850, and also provides a measure of safety by preventing objects from becoming entangled in the internal moving parts of laser radiating device 850. Inner dome 856 applies optical energy to the tissue of the subject via a light source or array of light sources (not shown).

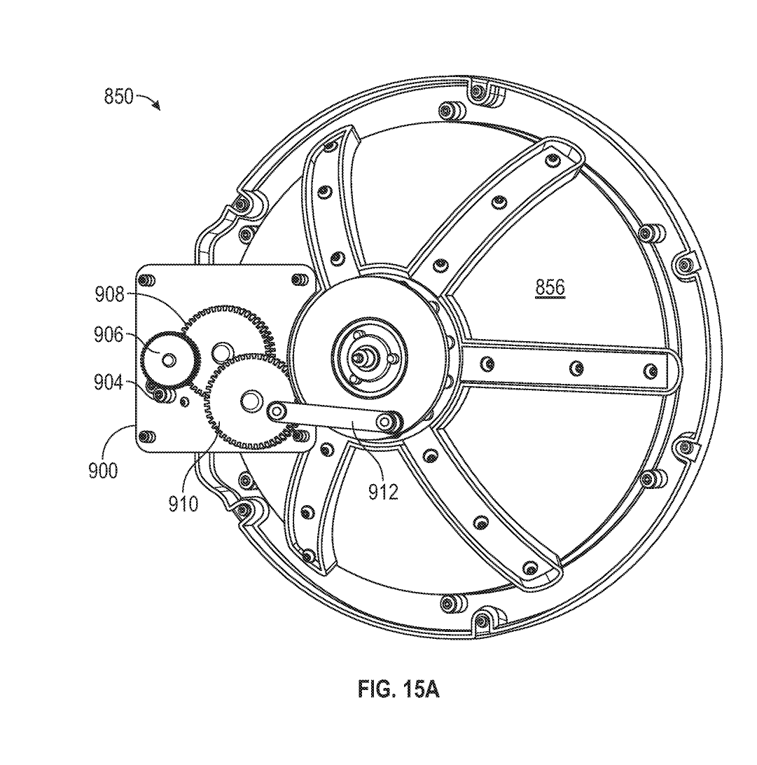

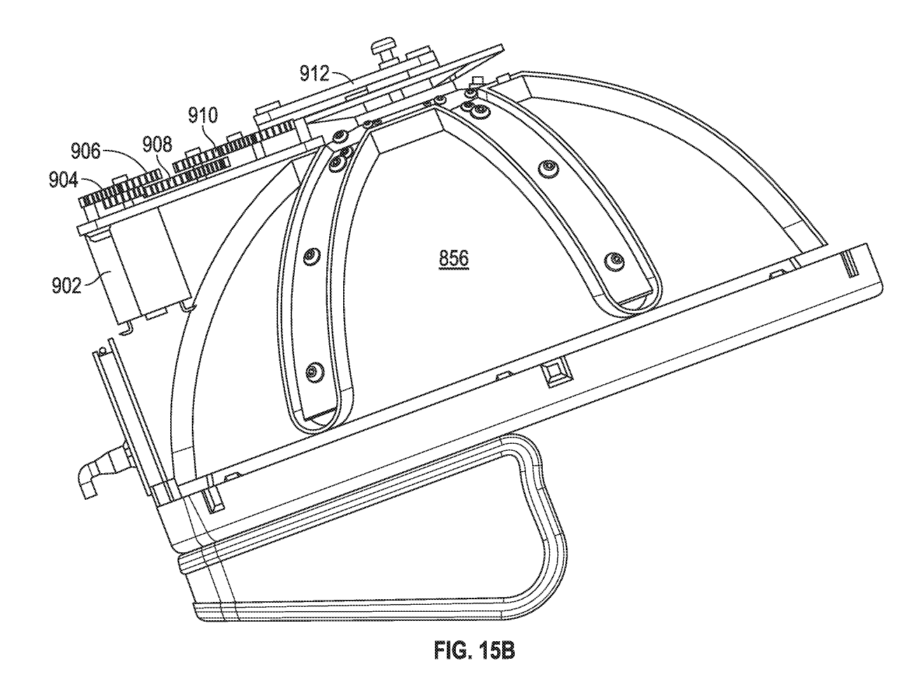

Upper housing 854 can have a generally-hemispherical shape. In other examples, the shape of the upper housing 854 is not limited to a hemispherical shape and can be, for example, triangular, rectangular, or flat. The operational mechanisms of laser radiating device 850 are enclosed within the upper anterior portion of upper housing 854. These include both electronic controls and mechanical actuation mechanisms, which are described in further detail below in connection with FIGS. 15A and 15B as well as similar components to the laser radiating devices discussed above. The upper anterior portion of upper housing 854 can include a vent 858, an access panel 860, a connector 862, and/or a control switch 864. Vent 858 allows heat to escape from upper housing 854, so as to prevent an overtemperature condition within laser radiating device 850. Additionally, vent 858 can allow air flow between internal the laser radiating device 850 and external the laser radiating device 850, for example, to provide aeration and prevent odors to develop. Access panel 860 can provide convenient access to certain operational controls and internal mechanisms without necessitating removal of upper housing 854. Connector 862 can provide communication between laser radiating device 850 and an external control unit, such as control unit 180 described above in connection with FIG. 6. Control switch 864 can control the flow of power to laser radiating device 850.

Inner dome 856 provides therapeutic light to the tissue of a subject, for example the scalp, via one or more light sources (not shown). The light sources employed may be any of the various types of light sources shown and described in the foregoing disclosure. The light may be applied in a specific pattern, or may be generally diffused evenly across the tissue. In at least one example, inner dome 856 can be rotatably mounted to frame 653 so as to allow inner dome 856 to rotate about a generally-vertical axis of rotation, thereby providing more even distribution of the applied light. In certain examples, inner dome 856 may rotate continuously is one direction. In other examples, inner dome 856 may oscillate back and forth about its axis of rotation. Additionally, in some examples, the inner dome 856 can be rotated to focus the radiated optical energy at the region to be treated.

Certain examples may include additional sensors, similar to sensors 11, 13 discussed above, which may include hair follicle sensors and blood flow sensors to measure hair growth parameters and provide closed-loop control of the process. A hair follicle sensor may be used to scan the tissue then the treatment can focus on the area with less hair density. A blood flow sensor can be used to detect the blood flow in subcutaneous tissue. Feedback from the blood flow sensor feedback can then be used to optimize the treatment time and laser power level. Additionally, the sensors can be utilized to track the progression of the treatment by measuring the number of hair follicles and/or blood flow before treatment and following up with measurements of hair follicles and/or blood flow during and/or after treatment. Areas around the regions to be treated may acceptable hair growth parameters and may not need treatment.

In certain examples, the unit may include multiple light sources. In certain such examples, individual light sources may be selectively turned on or off or the power level varied between light sources.

FIGS. 15A and 15B show a top and side view, respectively, of the laser radiating device 850 shown in FIGS. 14A-14D, as it appears with the top cover removed, so as to reveal the rotational mechanism for the inner dome 856.

As seen in FIGS. 15A and 15B, the motion of inner dome 856 is controlled by a motor-driven gear-and-crank mechanism attached to a geartrain frame 900. The mechanism comprises motor 902, secured to frame 900, having a motor pinion 904 connected to its rotor. Motor pinion 904 drives the driven spur gear of first intermediate spur assembly 906, which is also rotatably secured to frame 900. A first intermediate pinion, secured to the bottom of the driven spur gear of the first intermediate spur assembly 906, drives the driven spur gear of second intermediate spur assembly 908, also rotatably secured to frame 900. A second intermediate pinion, secured to the top of the driven spur gear of the second intermediate spur assembly, drives the crank spur gear 910. Crank spur gear 910, secured to the frame 900, is connected to the inner dome 856 by connecting rod 912, thereby allowing a control unit, such as control unit 180, to control the position of inner dome 856 via motor 902. While FIGS. 15A and 15B illustrate one exemplary mechanism to move components of the laser radiating device 850, other suitable mechanisms may be utilized without departing from the scope of the invention.

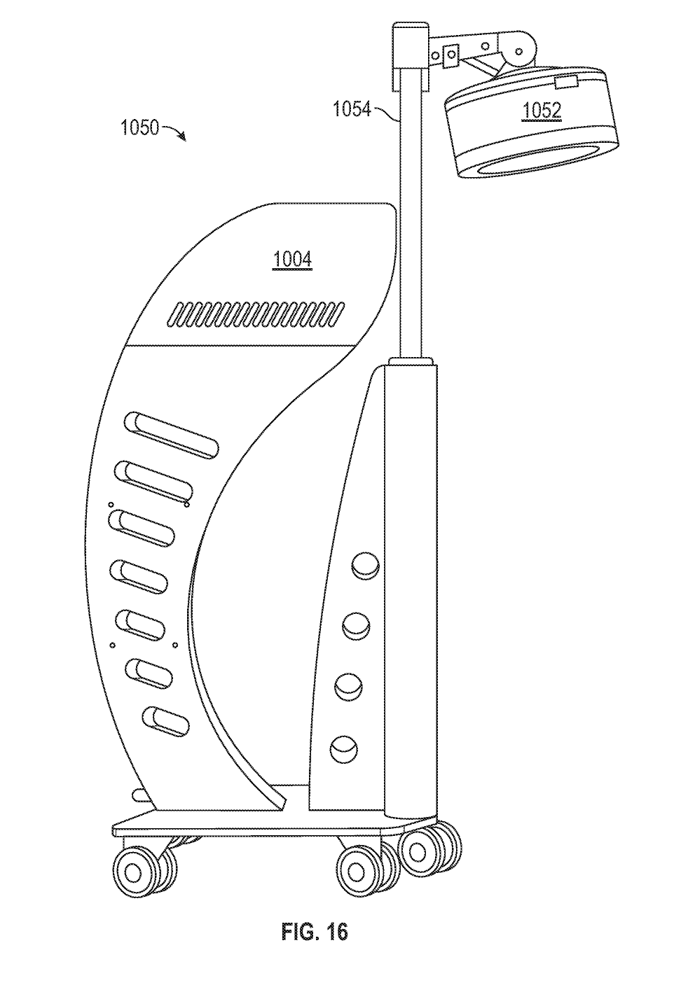

FIG. 16 shows a side view of a laser radiating device 1050, as a freestanding light application device according to the present disclosure. Device 1050 is shown comprising a stationary bonnet 1052 provided for surrounding and covering a patient's head, in a manner resembling a well-known hair dryer. Bonnet 1052 may be supported on a cantilevered support 1054 to allow the bonnet 1052 to be positioned over and about the head of a patient while maintaining a non-contact spacing between the interior of the bonnet 1052 and the scalp. Device 1050 further includes a control unit 1054, which may operate in a similar manner to controller 180, described above.

The patient's head may optionally be supported by an external chair having a neck rest (not shown) so that spacing between the scalp and the interior of the bonnet 1052 may be maintained. The bonnet 1052 may provide stable support for an inner dome therein, with the inner dome being actuated for rotation by a motor, as described above.

In the embodiment shown, a wiring harness may be routed through a hollow interior of the cantilevered support 1054 for convenience and to protect the wiring harness from snagging or damage. However, the wiring harness may also be attached directly to the bonnet 1052 by means of a coiled cable, a bundle of bound wires, or other suitable connection.

The laser radiating device 1050 may incorporate many or all of the features described above in connection with the other examples above. Generally, the laser radiating device 1050 will make use of an array of light sources, as described above. In some examples, the individual light sources may be selectively turned on and off, and the power output of each light source may be selectively controlled. By controlling the rotational position of the light sources, and selectively varying the pattern, the device has the capability to treat specific, programmable regions for specific, programmable periods of time using a variable and programmable laser power for hair growth.

As described above in connection with the other disclosed embodiments, the device 1050 may incorporate sensors, such as hair follicle sensors and blood flow sensors, to measure hair growth parameters and add feedback to better control the treatment parameters. A hair follicle sensor may be used to scan the tissue for hair density, allowing the device to focus treatment on areas with lower hair density. A blood flow sensor can be used to detect and measure the blood flow in subcutaneous tissue. The blood flow sensor feedback can then be used to optimize the treatment time and laser power level.

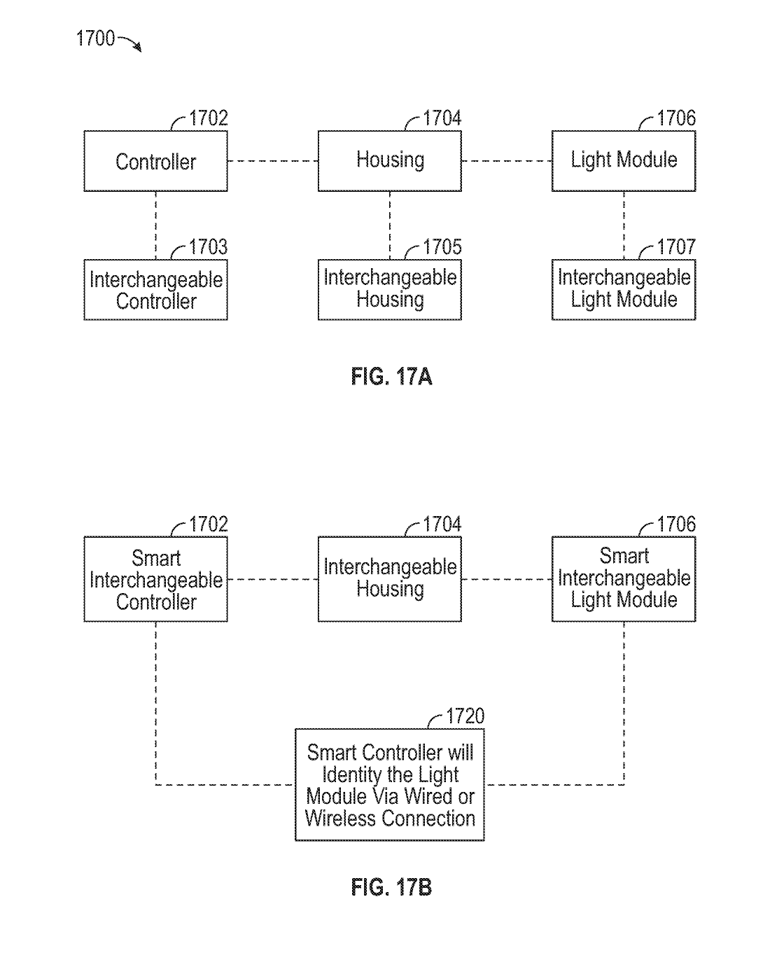

In at least one example, the system, for example any of the above systems and laser radiating devices, can include interchangeable elements for application of laser light. This modularity allows greater flexibility in the choice of components used in different therapeutic treatments. FIGS. 17A and 17B are diagrams of a modular laser system.

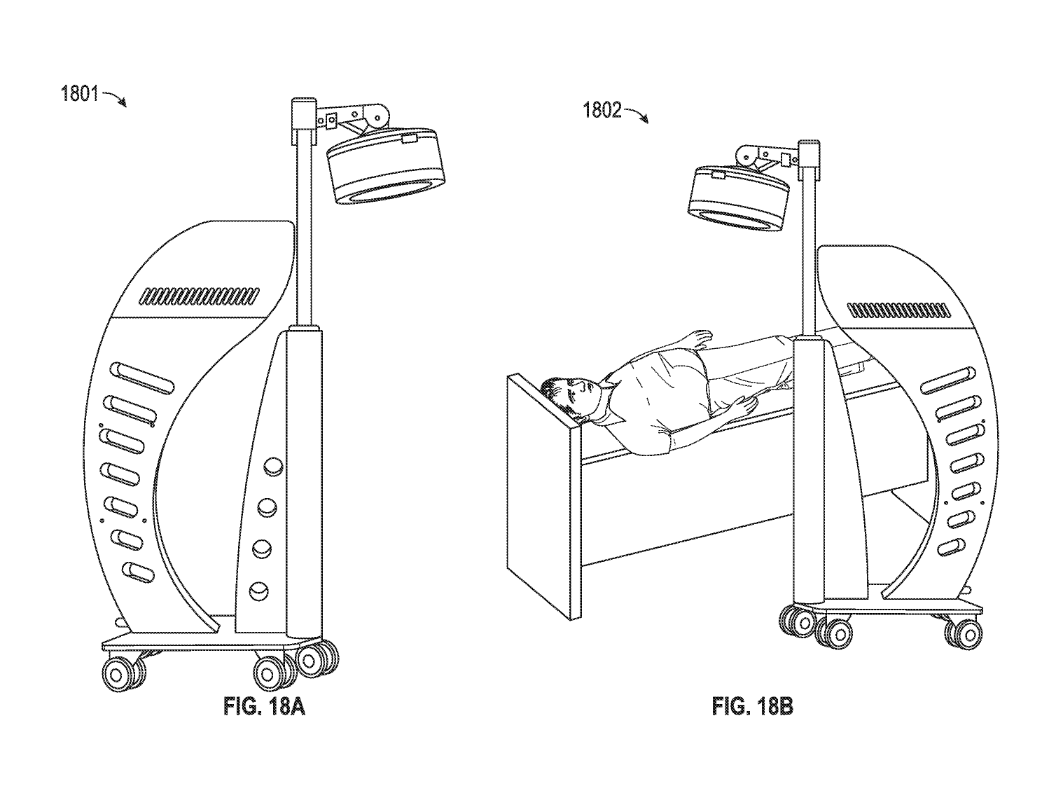

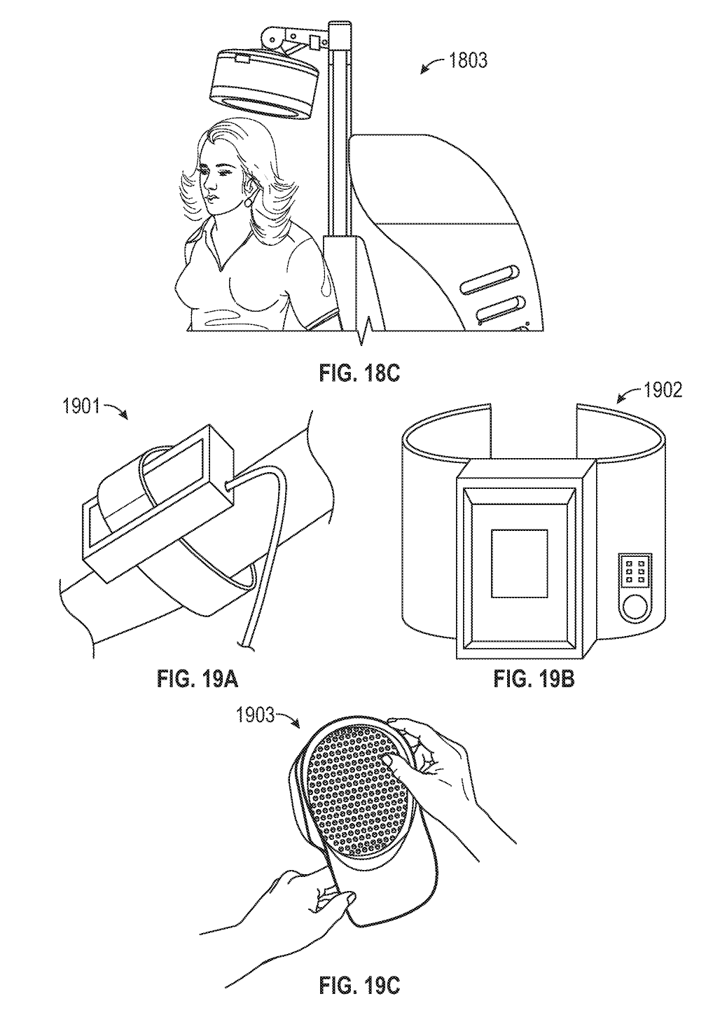

Once the smart control unit 1702 identifies the light module 1706 connected to the housing 1704, 1705 the smart control unit 1702 can load appropriate control parameters and/or appropriate graphical user interface (GUI). For example, for hair growth laser—a hair laser module 1707 might have 48 laser diodes, 72 laser diodes or 96 diodes. When different hair laser modules 1707 are connected to the smart control unit, the control unit 1702 can re-configure itself so the laser diode power output can be maintain at the same level. This modular reconfiguring can also be applied to different light sources with different wavelengths and power outputs for different therapeutic applications, for example hair laser, pain management laser, skin therapy laser, and/or acupressure. For example, at block 1720, when a new light module 1707 is connected to the smart control unit 1702, the control unit 1702 will re-configure itself to load specific software with specific user interface and control parameters to control the light module 1707. The model can be applied to both clinical and home devices, as shown in FIGS. 18A-18C and 19A-19C, respectively. FIG. 18A illustrates an exemplary skin care light therapy system 1801 which can be modified to include modules for a paint management system 1802 as illustrated in FIG. 18B and/or to include modules for a hair growth laser radiating device 1803 as illustrated in FIG. 18C. FIG. 19A illustrates a wearable device 1901 which can be modified to include modules for a paint management system 1902 as illustrated in FIG. 19B and/or to include modules for a hair growth laser radiating device 1903 as illustrated in FIG. 19C.

In some examples, the devices can include a motor communicatively coupled with the control unit 1702 that can rotate one or more of the modules, for example as discussed above in FIG. 11, to direct the treatment to the region to be treated for a predetermined period of time. Additionally or alternately, the light sources can be selectively turned on or off by the control unit 1702 to focus the radiated optical energy within the boundary of the region to be treated. Additionally or alternately, the radiated wavelength and/or the intensity of the optical energy can be selectively controlled by the control unit 1702. In at least one example, the devices can include sensors, similar to sensors 11, 13 discussed above, which may include hair follicle sensors and blood flow sensors to measure hair growth parameters and provide closed-loop control of the process. A hair follicle sensor may be used to scan the tissue then the treatment can focus on the area with less hair density. A blood flow sensor can be used to detect the blood flow in subcutaneous tissue. Feedback from the blood flow sensor feedback can then be used to optimize the treatment time and laser power level. Additionally, the sensors can be utilized to track the progression of the treatment by measuring the number of hair follicles and/or blood flow before treatment and following up with measurements of hair follicles and/or blood flow during and/or after treatment. Areas around the regions to be treated may acceptable hair growth parameters and may not need treatment.

In addition to having direct benefits itself, in at least one example, laser therapy can also potentiate and enhance the effectiveness of other modalities of treatment for hair growth, for example those involving the injection of bioactive compounds.

One such bioactive compound is platelet-rich plasma (PRP). PRP is a concentrate of platelet-rich plasma protein derived from whole blood that has been centrifuged to remove the red blood cells. Human blood is comprised of 93% red blood cells, 6% white blood cells, 1% platelets and plasma. Though platelets are best known for their function of blood clotting they are also a critical component in injury healing.

PRP has a greater concentration of growth factors than whole blood and is used to accelerate healing. In typical preparations PRP contains a concentration of platelets 3-5 times physiological levels. PRP contains several growth factors and cytokines that can stimulate healing of soft tissue. Growth factors found in PRP can include:

platelet-derived growth factor
transforming growth factor beta
fibroblast growth factor
insulin-like growth factor 1
insulin-like growth factor 2
vascular endothelial growth factor
epidermal growth factor

### Interleukin 8

keratinocyte growth factor
connective tissue growth factor

In dermatological applications, PRP is used for alopecia (hair loss), wound healing, and skin rejuvenation and is injected into the target tissue. PRP is activated with DNA activators (thrombin) and enriched with calcium ions (e.g. calcium chloride). Activated PRP is injected into the target area in order to stimulate healing or hair growth. The goal of PRP therapy is to maximize the number of platelets while minimizing the number of red blood cells in a solution that is injected into the injured area(s).

The four categories of PRP preparation are based on leukocyte and fibrin content: leukocyte-rich PRP (L-PRP), leukocyte reduced PRP (P-PRP), leukocyte platelet-rich fibrin, and pure platelet-rich fibrin (PRF).

Platelet-rich fibrin (PRF) or leukocyte- and platelet-rich fibrin (L-PRF) is a second-generation PRP in which autologous (i.e. obtained from the same individual) platelets and leucocytes are present in a complex fibrin matrix to accelerate the healing of soft and hard tissue.

Unlike other platelet concentrates, the production of PRF does not require any gelifying agent. Anticoagulant is not used during the centrifugation of the blood, and in fact coagulation is important for the formation of the fibrin matrix that traps the platelets and growth factors during production of PRF. Some of the advantages of PRF over other PRPs can include simpler preparation protocols, not needing to biochemically handle the blood, no need for bovine thrombin and anticoagulants, favorable healing due to slow polymerization, the ability of PRF to release growth factors in a controlled way, and more efficient cell migration and proliferation.

PRF has predominantly been used as a tissue-engineering scaffold in dentistry and endodontics. However, recent research has shown its promise for promoting hair growth. One possible mechanism is PRF's ability to induce dermal angiogenesis, thereby improving blood flow to hair follicles. PRF injections have been successfully implemented as a method of treating alopecia both as a standalone therapy and as an adjunct to hair follicular unit transplantation.

Another promising method of treatment for alopecia is stem cell injection. Stem cells are undifferentiated biological cells that can differentiate into specialized cells and divide to produce more stem cells. In adult organisms, stem cells and progenitor cells act as a repair system for the body, replenishing adult tissues. For example, accessible sources of autologous adult stem cells in humans can include bone marrow, adipose tissue, and blood.

In the skin, epithelial stem cells (EpSCs) in the hair follicle contribute not only to the generation of a new hair follicle with each hair cycle but also to the repair of the epidermis during wound healing. Hair follicle EpSCs reside in a specialized microenvironment called the bulge, a region at or near the base of the non-cycling portion of each hair follicle.

In cases of alopecia, stem cells (particularly adipose-derived) injected intradermally have the capacity to differentiate into mesochymal lineage cells. Additionally, stem cells can secrete growth factors that aid in restoration of hair growth.

In at least one example, the present system can employ laser light therapy in conjunction with intradermal injection of bioactive compounds to promote hair growth. The bioactive compounds used can be PRP, PRF, stem cells, individually or in combination. The laser light treatment can employ any of the laser devices described above. The laser wavelength employed can be in the range of 735 nm-10,000 nm. In at least one example, the laser wavelength can be about 800 nm to about 1350 nm. More specifically, best results appear to be achieved at 1064 nm, 1350 nm, 1550 nm, and 3150 nm. These specific wavelengths appears to have the greatest absorption as well as penetration into the dermis.

The precise mechanism by which PRP promotes hair growth is still not entirely understood. Activated PRP may increase the proliferation of human derma papilla (DP) cells through an increase in phosphorylation of extracellular signal-regulated kinases (ERK) and protein kinase B (also known as Akt). Although ERK signaling contributes to the regulation of cell growth, Akt can have anti-apoptotic effects in many cell types.

Activated PRP may also increase levels of the anti-apoptotic protein Bcl-2, protecting cells from apoptosis. Furthermore, activated PRP appears to contribute to the formation of hair epithelium and the differentiation of stem cells into hair follicle (HF) cells, through an upregulation of b-catenin, strongly expressed in the bulge region of the human anagen HF. It also prolongs the anagen phase of the hair cycle through an increase in expression of fibroblast growth factor-7.

Apart from these mechanisms, PRP may increase proliferation of epidermal and HF bulge cells, revealed by an increase in Ki-67 (marker for cell proliferation) in androgenetic alopecia (AGA). In alopecia areata (AA) too, an increase in Ki-67 has been noted, and PRP may act as a potent anti-inflammatory agent, suppressing release of inflammation cytokines.

When using laser irradiation in combination bioactive compounds for hair growth, less power is used per diode to enhance blood flow to the treated tissue. For example, 60 mw per diode might be used instead of 90 mw. By increasing the blood flow to the total hair follicle, the Ki-67 factor may increase hair growth.

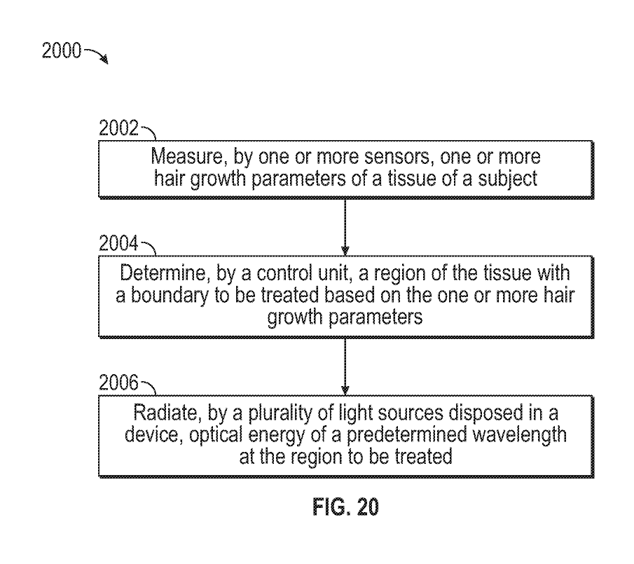

Referring to FIG. 20, a flowchart is presented in accordance with an example embodiment. The method 2000 is provided by way of example, as there are a variety of ways to carry out the method. The method 2000 described below can be carried out using the configurations illustrated in FIGS. 1-19C, for example, and various elements of these figures are referenced in explaining example method 2000. Each block shown in FIG. 20 represents one or more processes, methods or subroutines, carried out in the example method 2000. Furthermore, the illustrated order of blocks is illustrative only and the order of the blocks can change according to the present disclosure. Additional blocks may be added or fewer blocks may be utilized, without departing from this disclosure. The example method 2000 can begin at block 2002.

At block 2002, one or more hair growth parameters of a tissue of a subject is measured by one or more sensors. The sensors can include a hair follicle sensor and/or a blood flow sensor. Accordingly, the hair growth parameters can include number of hair follicles and/or blood flow.

At block 2004, a region of the tissue with a boundary to be treated is determined based on the one or more measured hair growth parameters. The region to be treated can be determined by the number of hair follicles being less than a predetermined number and/or blood flow being less than a predetermined amount.

At block 2006, optical energy at a predetermined wavelength is radiated by a plurality of light sources disposed in a device at the region to be treated. The optical energy is radiated within the boundaries of the region. In at least one example, the wavelength can be between about 1000 nm and about 10,000 nm. In some examples, the wavelength can be between about 1300 nm and about 4000 nm. In some examples, the wavelength can be between about 1550 nm and about 3150 nm.

The device can include a plurality of modules. Each of the plurality of modules can include at least one of the plurality of light sources and at least one of the one or more sensors. In at least one example, each of the plurality of modules can include a control unit coupled with the light sources and the sensors, and a memory including instructions executable by the control unit. As such, the modules can independently function as well as provide modularity by replacement of modules.

Optical energy at a predetermined wavelength can be radiated by the light sources disposed on at least one of the modules which is positioned above the region to be treated. The device can also include a motor coupled with each of the plurality of modules. The motor can move at least one of the plurality of modules to position the at least one module above the region to be treated. The motor can be communicatively coupled with the control unit, and the control unit can instruct the motor to move the modules based on the measured hair growth parameters. In at least one example, the modules can be moved by rotation around an axis. Accordingly, the motor can move the appropriate module to focus treatment on the region to be treated.

The description of the present invention has been presented for purposes of illustration and description, and is not intended to be exhaustive or limited to the invention in the form disclosed. Many modifications and variations will be apparent to those of ordinary skill in the art. The example was chosen and described in order to best explain the principles of the invention, the practical application, and to enable others of ordinary skill in the art to understand the invention for various examples with various modifications as are suited to the particular use contemplated. It will be understood by one of ordinary skill in the art that numerous variations will be possible to the disclosed examples without going outside the scope of the invention as disclosed in the claims.

What is claimed is:

1. A device comprising:
one or more sensors operable to measure one or more hair growth parameters of a tissue of a subject;
a plurality of light sources operable to radiate optical energy at a predetermined wavelength to promote hair growth;
a control unit coupled with the one or more sensors and the plurality of light sources; and
a memory storing instructions executable by the control unit to:
receive, from the one or more sensors, the one or more measured hair growth parameters,
determine a region within a boundary to be treated based on the one or more measured hair growth parameters, and
radiate, by the plurality of light sources, optical energy at the predetermined wavelength at the region to be treated, wherein the optical energy is radiated within the boundaries of the region.

2. The device of claim 1, further comprising:
a plurality of modules, each of the plurality of modules including:
at least one of the plurality of light sources, and
at least one of the one or more sensors.

3. The device of claim 2, wherein the memory further includes instructions executable by the control unit to:
radiate, by the light sources disposed on at least one of the plurality of modules which is positioned above the region to be treated, the optical energy at the predetermined wavelength.

4. The device of claim 3, further comprising:
a motor coupled with each of the plurality of modules, wherein the memory further includes instructions executable by the control unit to:
move at least one of the plurality of modules to position the at least one of the plurality of modules above the region to be treated.

5. The device of claim 4, wherein the at least one of the plurality of modules is moved by rotation around an axis.

6. The device of claim 2, wherein each of the plurality of modules further includes:
a control unit coupled with the at least one of the plurality of light sources and the at least one of the one or more sensors; and
a memory including instructions executable by the control unit.

7. The device of claim 1, wherein the one or more hair growth parameters includes number of hair follicles and/or blood flow.

8. The device of claim 7, wherein the region to be treated is determined by the number of hair follicles being less than a predetermined number and/or blood flow less than a predetermined amount.

9. The device of claim 1, wherein the predetermined wavelength is between about 100 nm and about 10,000 nm.

10. The device of claim 1, wherein the predetermined wavelength is between about 550 nm and about 4000 nm.

11. A method comprising:
measuring, by one or more sensors, one or more hair growth parameters of a tissue of a subject;
determining, by a control unit, a region of the tissue with a boundary to be treated based on the one or more measured hair growth parameters; and
radiating, by a plurality of light sources disposed in a device, optical energy at a predetermined wavelength at the region to be treated, wherein the optical energy is radiated within the boundaries of the region.

12. The method of claim 11, wherein the device includes a plurality of modules, each of the plurality of modules including:
at least one of the plurality of light sources; and
at least one of the one or more sensors.

13. The method of claim 12, further comprising:
radiating, by the light sources disposed on at least one of the plurality of modules which is positioned above the region to be treated, the optical energy at the predetermined wavelength.

14. The method of claim 13, wherein the device further includes a motor coupled with each of the plurality of modules, the method further comprising:
moving, by the motor, at least one of the plurality of modules to position the at least one of the plurality of modules above the region to be treated.

15. The method of claim 14, wherein the at least one of the plurality of modules is moved by rotation around an axis.

16. The method of claim 12, wherein each of the plurality of modules further includes:
a control unit coupled with the at least one of the plurality of light sources and the at least one of the one or more sensors; and
a memory including instructions executable by the control unit.

17. The method of claim 11, wherein the one or more hair growth parameters includes number of hair follicles and/or blood flow.

18. The method of claim 17, wherein the region to be treated is determined by the number of hair follicles being less than a predetermined number and/or blood flow less than a predetermined amount.

19. The method of claim 11, wherein the predetermined wavelength is between about 100 nm and about 10,000 nm.

20. The method of claim 11, wherein the predetermined wavelength is between about 550 nm and about 4000 nm.
